(19) United States
(12) Patent Application Publication
(10) Pub. No.: US 2023/0295103 A1
(43) Pub. Date: Sep. 21, 2023

(54) AURONE DERIVATIVES AND USES THEREOF FOR CONTROLLING BACTERIA AND/OR FUNGI

(71) Applicants: UNIVERSITE D'AIX-MARSEILLE, MARSEILLE (FR); CENTRE NATIONAL DE LA RECHERCHE SCIENTIFIQUE, PARIS (FR); ECOLE CENTRALE DE MARSEILLE, MARSEILLE (FR); INSTITUT NATIONAL DE LA SANTE ET DE LA RECHERCHE MEDICALE, PARIS (FR); Etat Francais Représenté Par La Direction Centrale Du Service De Santé Des Armées, PARIS (FR)

(72) Inventors: MAXIME ROBIN, BOUC BEL AIR (FR); MARC MARESCA, VITROLLES (FR); HAMZA OLLEIK, TOULOUSE (FR); VALÉRIE PIQUE, MONTFOR SUR ARGENS (FR); JOSETTE PERRIER-VIRET, MARTIGUES (FR); JEAN-MICHEL BOLLA, MARSEILLE (FR); FABIENNE NEULAT-RIPOLL, LA NORVILLE (FR)

(21) Appl. No.: 18/014,762

(22) PCT Filed: Jul. 8, 2021
(86) PCT No.: PCT/EP2021/069047

§ 371 Date: Jan. 6, 2023

(30) Foreign Application Priority Data
Jul. 8, 2020 (EP) 20305783.1

(51) Int. Cl. C07D 307/83; C07D 407/12; C07D 455/03; A01P 1/00; A01P 3/00; A61P 31/04; A61P 31/10; A01N 43/12; A01N 43/16; A01N 43/42

(52) U.S. Cl. CPC C07D 307/83; C07D 407/12; C07D 455/03; A01P 1/00; A01P 3/00; A61P 31/04; A61P 31/10; A01N 43/12; A01N 43/16; A01N 43/42

(57) ABSTRACT

The present invention relates to aurones compounds of formula (I) and their uses for controlling a bacterium and/or a fungus. The invention also relates to the use of such compounds as phytoprotective and/or decontaminating and/or disinfectant agent and compositions comprising them.

### TECHNICAL FIELD

The present invention relates to the microbial and fungal fields, and more particularly to aurone derivatives and compositions comprising the same exhibiting antimicrobial and antifungal properties. Such derivatives are suitable for a large panel of applications, such as phytosanitary, phytoprotective, decontaminating, and disinfectant applications as well as human and veterinary medicine.

### TECHNICAL BACKGROUND

Flavonoids are natural products, including for instance flavones, chalcones, flavanones and derivatives thereof, which provide promising antibacterial activities. It further exists a minor subclass of flavonoids called “aurones” having the following scaffold:

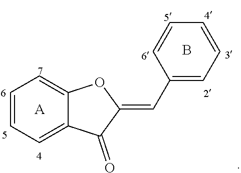

These aurones play a role of flower pigments, nectar guides or antioxidant. Among natural derivatives of aurones, three more specific compounds, namely, cephalocerone, hispidol, and hispidol-4′-O-β-D-glucoside comprising a hydroxy on the carbon 6 of the cycle A of the aurone scaffold have been identified as efficient agent against bacteria and fungus. More particularly, cephalocerone has been found to inhibit the growth of the Gram⁻ bacterium Erwina cacticida, and hispidol and hispidol-4′-O-β-D-glucoside to inhibit the fungal pathogen Phoma medicaginis. Accordingly, the diversity of aurone derivatives could be an interesting approach for the control of a pathogen such as a bacterium and/or a fungus.

At the beginning of 2019, Olleik et al. (European Journal of Medicinal Chemistry, 2019, 165, 133-141) have synthesized and evaluated aurone derivatives against some Gram+ and Gram− bacterial and fungal species. However, only the aurones substituted by a hydroxy in carbons 4 and 6 of the cycle A of the aurone scaffold have shown a first sign of efficacy.

It thus remains a need for developing safe or no toxic antimicrobial and/or antifungal products comprising aurones derivatives. More particularly, there is still a need to provide aurone derivatives exhibiting an improved efficacy against a large panel of bacterium and fungus.

### SUMMARY OF THE INVENTION

In this context, the inventors have provided aurone derivatives of formula (I) having a bactericidal activity against Gram+ and Gram− bacteria, including resistant strains, and a fungicidal activity, with a micromolar minimal inhibitory concentration (MIC), preferably lower than 100 μM. The inventors have also demonstrated the safety, i.e. the non-toxicity of the compounds of the formula (I) of the invention.

The present invention thus relates to a compound of formula (I), the salts and the stereoisomers thereof:

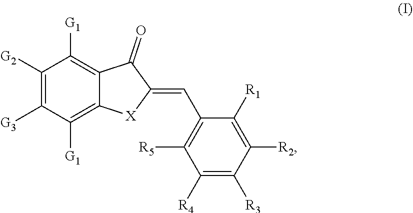

wherein:
X represents O, NH, S, or —CH₂—;
G₁ represents a hydrogen or a (C₁-C₆)alkyloxy;
G₂ represents a radical selected in the group consisting of:
   a hydrogen,
   a (C₁-C₆)alkyl optionally substituted by a —NR₆R₇ group with R₆ and R₇ being independently a hydrogen or a —COR₈ with R₈ being a (C₁-C₆)alkyl;
   a —NR₆R₇ group with R₆ and R₇ being independently a hydrogen, a —COR₈ with R₈ being a (C₁-C₆)alkyl, or an arginine residue;
   a carbamimidoyl-guanidine; and
   a —COOR₉ with R₉ being a hydrogen, a (C₁-C₁₂)alkyl, a geranyl, a farnesyl or a citronnellyl;
G₃ represents a radical selected in the group consisting of:
   a hydrogen,
   a hydroxy,
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
   with the proviso that when G₂ and G₃ represent a hydrogen, then:
R₁ and R₃ represent a benzyloxy, or
R₃ represents a —O-flavone optionally substituted by at least a (C₁-C₁₀)alkyloxy or a hydroxy, or a —CH₂-berberine; and
   with the proviso that said compound of formula (I) is not a compound selected in the group consisting of:
(Z)-N-(2-(4-methoxybenzylidene)-3-oxo-2,3-dihydrobenzofuran-5-yl)acetamide;
(Z)-N-(3-oxo-2-(3,4,5-trimethoxybenzylidene)-2,3-dihydrobenzofuran-5-yl)acetamide; and
(Z)-N-(2-benzylidene-3-oxo-2,3-dihydrobenzofuran-5-yl)acetamide.

Another object of the invention is a use of a compound of formula (I), the salts and the stereoisomers thereof:

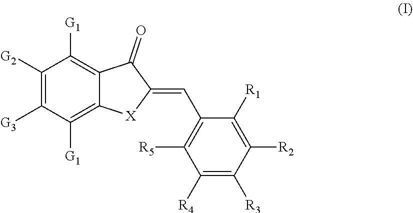

wherein:
X represents O, NH, S, or CH₂—;
G₁ represents a hydrogen or a (C₁-C₆)alkyloxy;
G₂ represents a radical selected in the group consisting of:
   a hydrogen,
   a (C₁-C₆)alkyl optionally substituted by a —NR₆R₇ group with R₆ and R₇ being independently a hydrogen or a —COR₈ with R₈ being a (C₁-C₆)alkyl;
   a —NR₆R₇ group with R₆ and R₇ being independently a hydrogen, a —COR₈ with R₈ being a (C₁-C₆)alkyl, or an arginine residue;
   a carbamimidoyl-guanidine; and
   a —COOR₉ with R₉ being a hydrogen, a (C₁-C₁₂)alkyl, a geranyl, a farnesyl or a citronnellyl;
G₃ represents a radical selected in the group consisting of:
   a hydrogen,
   a hydroxy,
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₄ represents a hydrogen, a nitro, or a (C₁-C₆)alkyl;
   R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
   with the proviso that when G₂ and G₃ represent a hydrogen, then:
R₁ and R₃ represent a benzyloxy, or
R₃ represents a —O-flavone optionally substituted by at least a (C₁-C₁₀)alkyloxy or a hydroxy, or a —CH₂-berberine;
   or a compound D10: (Z)-2-(4-methoxybenzylidene)benzofuran-3(2H)-one;
   for controlling a bacterium and/or a fungus.

In a particular embodiment, R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl.

In a preferred embodiment, a compound of formula (I) is such that:
G₂ represents a hydrogen, or a —NR₆R₇ group with R₆ and R₇ being independently a hydrogen or a —COR₈ with R₈ being a (C₁-C₆)alkyl, preferably a methyl; and
G₃ represents a hydrogen, or a —NR₆R₇ group with R₆ and R₇ being independently a hydrogen or a —COR₈ with R₈ being a (C₁-C₆)alkyl, preferably a methyl;
   with the proviso that when one of G₂ and G₃ represents a —NR₆R₇ group with R₆ and R₇ being independently a hydrogen or a —COR₈ with R₈ being a (C₁-C₆)alkyl, preferably a methyl, then the other represents a hydrogen.

In a further particular embodiment, R₃ represents a —O-flavone optionally substituted by at least a (C₁-C₁₀)alkyloxy or a hydroxy, or a —CH₂-berberine.

In a further particular embodiment, G₂ represents COOH.

In a further particular embodiment, R₃ and R₄ represent a benzyloxy.

Preferably, a compound of formula (I) is selected in the group consisting of:
A1: (Z)-N-(2-(2′-methoxybenzylidene)-3-oxo-2,3-dihydrobenzofuran-5-yl)acetamide;
A2: (Z)-N-(2-(3-methoxybenzylidene)-3-oxo-2,3-dihydrobenzofuran-5-yl)acetamide;
A7: (Z)-N-(2-(3-(benzyloxy)benzylidene)-3-oxo-2,3-dihydrobenzofuran-5-yl)acetamide;
A9: (Z)-5-amino-2-(3-(benzyloxy)benzylidene)benzofuran-3(2H)-one;
A112: (Z)-N-(3-oxo-2-(4-phenoxybenzylidene)-2,3-dihydrobenzofuran-5-yl)acetamide;
B1: (Z)-5-amino-2-(3-phenoxybenzylidene)benzofuran-3(2H)-one;
B4: (Z)-N-(2-(3-isopropoxybenzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
B10: (Z)-N-(2-(3-fluorobenzylidene)-3-oxo-2,3-dihydrobenzofuran-5-yl)acetamide;
B11: (Z)-5-amino-2-(2-fluorobenzylidene)benzofuran-3(2H)-one;
C4: (Z)-4-((5-acetamido-3-oxobenzofuran-2(3H)-ylidene)methyl)benzoic acid;
C8: (Z)-N-(7-nitro-3-oxo-2-(3-phenoxybenzylidene)-2,3-dihydrobenzofuran-5-yl)acetamide;
C10: (Z)-2-benzylidene-6-hydroxy-4-methoxy-5,7-dimethylbenzofuran-3(2H)-one;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
D2: (Z)-5-amino-2-(3,4,5-trimethoxybenzylidene)benzofuran-3(2H)-one;
D3: (Z)-N-(3-oxo-2-(2,4,5-trimethoxybenzylidene)-2,3-dihydrobenzofuran-5-yl)acetamide;
E5: (Z)-6-(4-((3-oxobenzofuran-2(3H)-ylidene)methyl)phenoxy)-2-phenyl-4H-chromen-4-one; and
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide;
AD-1-44: (Z)-N-(2-(3,4-bis(benzyloxy)benzylidene)-3-oxo-2,3-dihydrobenzofuran-5-yl)acetamide;
MR1065: (Z)-2-(3,4-bis(benzyloxy)benzylidene)-3-oxo-2,3-dihydrobenzofuran-5-carboxylic acid;
MR1076: (Z)-6-(4-((3-oxobenzofuran-2(3H)-ylidene)methyl)phenoxy)hexanoic acid;
MR1120: (Z)-ethyl 2-(3,4-bis(benzyloxy)benzylidene)-3-oxo-2,3-dihydrobenzofuran-5-carboxylate; and
MR1121: (Z)-decyl 2-(3,4-bis(benzyloxy)benzylidene)-3-oxo-2,3-dihydrobenzofuran-5-carboxylate.

Preferably said compound is selected in the group consisting of:
MR1065: (Z)-2-(3,4-bis(benzyloxy)benzylidene)-3-oxo-2,3-dihydrobenzofuran-5-carboxylic acid
A7: (Z)-N-(2-(3-(benzyloxy)benzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
E5: (Z)-6-(4-((3-oxobenzofuran-2(3H)-ylidene)methyl)phenoxy)-2-phenyl-4H-chromen-4-one; and
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide.

Preferably, a use of a compound of formula (I) for controlling a bacterium and/or a fungus is selected in the group consisting of:
A1: (Z)-N-(2-(2′-methoxybenzylidene)-3-oxo-2,3-dihydrobenzofuran-5-yl)acetamide;
A2: (Z)-N-(2-(3-methoxybenzylidene)-3-oxo-2,3-dihydrobenzofuran-5-yl)acetamide;
A7: (Z)-N-(2-(3-(benzyloxy)benzylidene)-3-oxo-2,3-dihydrobenzofuran-5-yl)acetamide;
A9: (Z)-5-amino-2-(3-(benzyloxy)benzylidene)benzofuran-3(2H)-one;
A12: (Z)-N-(3-oxo-2-(4-phenoxybenzylidene)-2,3-dihydrobenzofuran-5-yl)acetamide;
B1: (Z)-5-amino-2-(3-phenoxybenzylidene)benzofuran-3(2H)-one;
B4: (Z)-N-(2-(3-isopropoxybenzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
B10: (Z)-N-(2-(3-fluorobenzylidene)-3-oxo-2,3-dihydrobenzofuran-5-yl)acetamide;
B11: (Z)-5-amino-2-(2-fluorobenzylidene)benzofuran-3(2H)-one;
C4: (Z)-4-((5-acetamido-3-oxobenzofuran-2(3H)-ylidene)methyl)benzoic acid;
C8: (Z)-N-(7-nitro-3-oxo-2-(3-phenoxybenzylidene)-2,3-dihydrobenzofuran-5-yl)acetamide;
C10: (Z)-2-benzylidene-6-hydroxy-4-methoxy-5,7-dimethylbenzofuran-3(2H)-one;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
D1: (Z)-N-(3-oxo-2-(3,4,5-trimethoxybenzylidene)-2,3-dihydrobenzofuran-5-yl)acetamide;
D2: (Z)-5-amino-2-(3,4,5-trimethoxybenzylidene)benzofuran-3(2H)-one;
D3: (Z)-N-(3-oxo-2-(2,4,5-trimethoxybenzylidene)-2,3-dihydrobenzofuran-5-yl)acetamide;
D8: (Z)-N-(2-benzylidene-3-oxo-2,3-dihydrobenzofuran-5-yl)acetamide;
D10: (Z)-2-(4-methoxybenzylidene)benzofuran-3(2H)-one;
E5: (Z)-6-(4-((3-oxobenzofuran-2(3H)-ylidene)methyl)phenoxy)-2-phenyl-4H-chromen-4-one;
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide;
AD-1-44: (Z)-N-(2-(3,4-bis(benzyloxy)benzylidene)-3-oxo-2,3-dihydrobenzofuran-5-yl)acetamide;
AD-1-61: (2Z)-2-(3-methoxybenzylidene)-3-oxo-2,3-dihydro-1-benzofuran-5-carboxylic acid
AD-1-62: (2Z)-2-[4-(dimethylamino)benzylidene]-3-oxo-2,3-dihydro-1-benzofuran-5-carboxylic acid;
MR1065: (Z)-2-(3,4-bis(benzyloxy)benzylidene)-3-oxo-2,3-dihydrobenzofuran-5-carboxylic acid;
MR1076: (Z)-6-(4-((3-oxobenzofuran-2(3H)-ylidene)methyl)phenoxy)hexanoic acid;
MR1120: (Z)-ethyl 2-(3,4-bis(benzyloxy)benzylidene)-3-oxo-2,3-dihydrobenzofuran-5-carboxylate; and
MR1121: (Z)-decyl 2-(3,4-bis(benzyloxy)benzylidene)-3-oxo-2,3-dihydrobenzofuran-5-carboxylate.

Preferably said compound is selected in the group consisting of:
MR1065: (Z)-2-(3,4-bis(benzyloxy)benzylidene)-3-oxo-2,3-dihydrobenzofuran-5-carboxylic acid;
A7: (Z)-N-(2-(3-(benzyloxy)benzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
E5: (Z)-6-(4-((3-oxobenzofuran-2(3H)-ylidene)methyl)phenoxy)-2-phenyl-4H-chromen-4-one; and
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide.

In a particular aspect, said bacterium is a Gram⁺ bacterium, a Gram⁻ bacterium, or a Mycobacterium. Preferably, Gram⁺ bacterium is selected in the group consisting of: Bacillus cereus, Bacillus subtilis, Clostridium botulinum, Clostridium coccoides, Clostridium difficile, Clostridium perfringens, Clostridium propionicum, Enterococcus faecalis, Lactococcus lactis, Listeria monocytogenes, Micrococcus luteus, Propionibacterium acnes, Staphylococcus aureus, Streptococcus pyogenes, and Streptomyces roietensis. Preferably, Gram⁻ bacterium is selected in the group consisting of: Acinetobacter baumannii, Bacteroides thetaioataomicron, Burkholderia cepacia, Burkholderia thailandensis, Burkholderia mallei, Burkholderia pseudomallei, Citrobacter farmer, Citrobacter rodentium, Escherichia coli, E. coli EHEC, Helicobacter pylori, Klebsiella pneumoniae, Klebsiella variicola, Pseudomonas aeruginosa, Pseudomonas syringae, Salmonella enterica, Shigella flexneri, and Vibrio alginolyticus.

In a more particular aspect, said bacteria is an antibiotic resistant bacterium, preferably an antibiotic resistant Gram⁺ bacterium, more preferably selected in the group consisting of: B. subtilis Nisin R, and S. aureus Methicillin R, or preferably an antibiotic resistant Gram⁻ bacterium, preferably Burkholderia cepacia, Burkholderia thai, Burkholderia mallei and Burkholderia pseudomallei.

In a further aspect, said fungus is selected in the group consisting of: Candida albicans, Metarhizium anisopliae, Beauveria feline, Aspergillus flavus, Fusarium graminearum, Fusarium verticillioides, Penicillium verrucosum, Fusarium oxysporum, Aspergillus niger, Stachybotrys chartarum, Aspergillus ochraceus, Microdochium bolleyi, Microdochium majus, and Microdochium nivale.

A further object of the invention is a use of a compound as defined herein as a phytoprotective and/or decontaminating and/or disinfectant agent. Particularly, said phytoprotective and/or decontaminating and/or disinfectant agent is applied on a surface or an inert object. More particularly, said phytoprotective and/or decontaminating and/or disinfectant agent is applied in combination with at least an anti-microbial and/or anti-fungal agent.

A further object of the invention is a phytoprotective or decontaminating or disinfectant composition comprising a compound as defined herein. In a particular embodiment, said composition further comprises an anti-microbial and/or anti-fungal agent.

Another object of the invention is a process for the decontamination or the disinfection of a surface or an inert object comprising a step of applying a phytoprotective or decontaminating or disinfectant composition as defined herein on a surface or an inert object infested or suspected to be infested by a bacterium and/or a fungus.

Another object of the invention is a kit comprising a compound as defined herein, and a further anti-microbial and/or anti-fungal agent as a combined preparation for simultaneous, separate, or sequential use, in particular for controlling a bacterium and/or a fungus.

The present invention also relates to a compound as defined herein for use as a medicine or a drug. The present invention also relates to a compound as defined herein for use for preventing and/or treating a bacterial infection and/or a fungal infection, preferably for preventing and/or treating bacterial infection and/or fungal infection caused by a bacterium and/or a fungus as defined herein. The present invention also relates to a compound as defined herein for use as an antibiotic or as an antifungal medication,

The present invention further relates to a pharmaceutical composition comprising a compound as defined herein, and a pharmaceutically acceptable excipient. Another object of the invention also concerns a pharmaceutical composition as defined herein, for use for preventing and/or treating a bacterial infection and/or a fungal infection.

### FIGURES

FIG. 1: Antimicrobial effect of the compounds of the invention on reference bacterial strains: E. coli (ATCC 8739), B. subtilis (ATCC 6633), P. aeruginosa (ATCC 9027), S. aureus (ATCC 6538P), H. pylori (ATCC43504), and antifungal effect of the compounds of the invention on reference fungal strain: C. albicans (ATCC 200498). Antimicrobial activity aurones was evaluated by determination of their minimal inhibitory concentration (MIC) using two-fold serial dilutions in liquid media following the National Committee of Clinical Laboratory Standards NCCLS, 1997 for bacteria and NCCLS M27-A and M38-P for fungi.

FIG. 2: Antimicrobial and antifungal effects of the particular compounds of the invention A7, B5, C11, C12, and E5 on reference bacterial and fungal strains including strains resistant to antibiotic. Antimicrobial activity aurones was evaluated by determination of their minimal inhibitory concentration (MIC) using two-fold serial dilutions in liquid media following the National Committee of Clinical Laboratory Standards NCCLS, 1997 for bacteria and NCCLS M27-A and M38-P for fungi.

FIG. 3: Antimicrobial effect of the compounds of the invention on Burkholderia strains. Antimicrobial activity aurones was evaluated by determination of their minimal inhibitory concentration (MIC) using two-fold serial dilutions in liquid media following the National Committee of Clinical Laboratory Standards NCCLS, 1997.

FIG. 4: Antibacterial effect of the compounds of the invention on bacterial plant pathogens Pseudomonas syringae (DSM 10604), and Streptomyces roietensis (DSM 101729). Antimicrobial activity aurones was evaluated by determination of their minimal inhibitory concentration (MIC) using two-fold serial dilutions in liquid media following the National Committee of Clinical Laboratory Standards NCCLS, 1997.

FIG. 5: Evaluation of the toxicity of compounds of the invention on different human cells. Effect of compounds according to the invention on cell viability was measured using resazurin assay after 48 h exposure to increasing concentrations of compounds. IC50 values (i.e. concentration of compounds causing a 50% decrease in the cell viability) were calculated from plotted data using Graph Pad Prism and are expressed in μM. Cells used were: human kidney cell line A498 (ATCC® HTB-44), human normal lung epithelial cells BEAS-2B (ATCC® CRL-9609), human intestinal cell line Caco-2 (ATCC® HTB-37), human normal epidermal keratinocytes HaCaT, human liver cell line HepG2 (ATCC® HB-8065), human normal vascular endothelial cells HUVEC, human normal lung fibroblast IMR90 cells (ATCC® CCL186) and human gastric cell line N87 (ATCC® CRL-5822).

FIG. 6: Therapeutic index or safety factor of the particular compounds of the invention A7, B5, C11, C12, and E5. Therapeutic index (TI) or safe factor (SF) were calculated by dividing the IC50 obtained for each cell type by the lowest MIC values observed for the same aurone.

FIGS. 7 and 8: Induction of resistance on B. mallei (FIG. 8) and B. pseudomallei (FIG. 7) by compounds A7, B1, B5, C4, C10, C11, C12, D1, D2, D10, and the control antibiotic doxycycline.

FIG. 9: Mutagenicity of tests compounds A7 (FIG. 9A), A9 (FIG. 9B), B1 (FIG. 9C), C10 (FIG. 9D), C11 (FIG. 9E), B5 (FIG. 9F), C12 (FIG. 9G), D1 (FIG. 9H), D10 (FIG. 9I), E5 (FIG. 9J), and F2 (FIG. 9K) by the in vitro micronucleus assay according to the OECD guideline No 487. BI: Binucleated cells; MONO: Mononucleated cells; CBPI: Cytokinesis-Blocked Proliferative Index; CI %: Cytostasis index expressed in percentage as compared to the control; MNC1, MNC-2: Micronucleated cell rates; MNC-M: Means of the micronucleated cell rates; P: probability of the chi-squared test (p<0.05: significant difference as compared to the control culture); NS: non-significant difference as compared to the control culture

FIG. 10: Antimicrobial effect of the particular compounds of the invention AD-1-44, AD-1-61, AD-1-62, MR1065 and MR1076 on reference bacterial strains: E. coli (ATCC 8739), B. subtilis (ATCC 6633), P. aeruginosa (ATCC 9027), S. aureus (ATCC 6538P), C. difficile (DSM 1296), and antifungal effect of the compounds of the invention on reference fungal strain: C. albicans (DSM 10697). Antimicrobial activity aurones was evaluated by determination of their minimal inhibitory concentration (MIC) using two-fold serial dilutions in liquid media following the National Committee of Clinical Laboratory Standards NCCLS, 1997 for bacteria and NCCLS M27-A and M38-P for fungi.

FIG. 11: Evaluation of the toxicity of compounds AD-1-44, AD-1-61, AD-1-62, MR1065 and MR1076 on human liver cell line HepG2 (ATCC® HB-8065). Effect of compounds according to the invention on cell viability was measured using resazurin assay after 48 h exposure to increasing concentrations of compounds. IC50 values (i.e. concentration of compounds causing a 50% decrease in the cell viability) were calculated from plotted data using Graph Pad Prism and are expressed in μM.

FIG. 12: Therapeutic index or safety factor of the particular compounds of the invention AD-1-44, AD-1-61, AD-1-62, MR1065 and MR1076. Therapeutic index (TI) or safe factor (SF) were calculated by dividing the IC50 obtained for each cell type by the lowest MIC values observed for the same compound.

### DETAILED DESCRIPTION OF THE INVENTION

### Definitions

According to the present invention, the terms below have the following meanings:

The terms mentioned herein with prefixes such as for example C₁-C₃, C₁-C₆ or C₁-C₁₀ can also be used with lower numbers of carbon atoms such as C₁-C₂, C₁-C₅, or C₂-C₅. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₂-C₆ is used, it means that the corresponding hydrocarbon chain may comprise from 2 to 6 carbon atoms, especially 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl.

The term “alkyloxy” or “alkoxy” corresponds to the alkyl group as above defined bonded to the molecule by an —O— (ether) bond. (C₁-C₃)alkyloxy includes methoxy, ethoxy, propyloxy, and isopropyloxy. (C₁-C₆)alkyloxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy or an isopropyloxy.

The term “halogen” corresponds to a fluorine, a chlorine, a bromine, or an iodine atom, preferably a fluorine.

The term “amino acid residue” corresponds to an amino acid attached to the rest of the molecule by the amino terminal group or the acido terminal group, preferably the acido terminal group. It can be represented by the formula NH₂—CH(R)—CO— in which R corresponds to the lateral chain of the amino acid. It includes any amino acid residue, such as an “arginine residue”, which can be represented by the following structure:

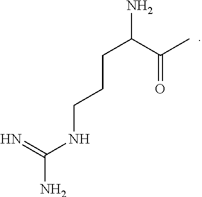

The term “carbamimidoyl-guanidine” can be represented by the structure:

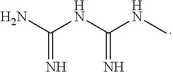

The term “geranyl” can be represented by the following structure:

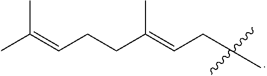 [SMILES: *C/C=C(\C)CCC=C(C)C]

The term “farnesyl” can be represented by the following structure:

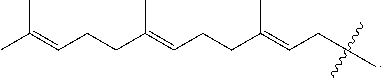 [SMILES: *C/C=C(\C)CC/C=C(\C)CCC=C(C)C]

The term “citronnellyl” can be represented by the following structure:

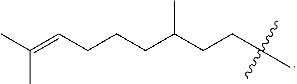 [SMILES: *CCC(C)CCCC=C(C)C]

The term “phenyloxy” corresponds to a —O-phenyl group. The term “benzyloxy” corresponds to a —O-benzyl group or a —O—CH₂-phenyl group.

The “flavone” group has the following structure:

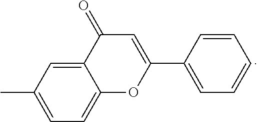 [SMILES: Cc1ccc2oc(-c3ccccc3)cc(=O)c2c1]

The “berberine” group has the following structure:

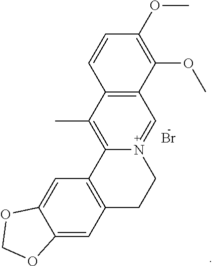

The expression “substituted by at least a” means that the radical is substituted by one or several groups of the list.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred embodiment of the invention, the stereoisomers include diastereoisomers and enantiomers.

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine.

As used herein, the terms “controlling”, or “control” a bacterium and/or a fungus includes the reduction, the elimination, the eradication, the killing, the decontamination, the prevention and/or the treatment of the bacterium and/or the fungus. In another embodiment, such terms further include slowing and/or stopping the development of the bacterium and/or the fungus.

As used herein, the terms “treatment”, “treat” or “treating” refer to any act intended to ameliorate the health status of patients such as therapy, prevention, prophylaxis and retardation of a disease, in particular an infection, preferably a bacterial infection and a fungal infection. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a composition having an effect, such as a phytoprotective effect, a decontaminating effect, a disinfectant effect, a therapeutical effect, such as an anti-infection effect, an anti-microbial effect, and an anti-fungal effect.

As used herein, a “surface” refers to any surface found at home or at work. For instance, it may be cited, without limitation, surfaces found in hospital, school, retirement home, shops, restaurant, laboratory (floor, walls, windows, bench). Ground in farm, street pavement, and playground may also be considered as a surface.

As used herein an “inert object” refers to any object used by human in his private or professional life. For instance, it may be cited, without limitation, instruments and objects used to cook, present in school, in laboratory, in retirement home, in hospital, in playground, and present at home (domestic objects). Inert object could may also include garbage bags, plastic wrap, textiles and carpet underlay.

As used herein, the term “phytoprotective” agent or “phytoprotective” composition refers to an agent or a composition that protects plants against any pathogens, such as bacteria and fungi.

As used herein, the term “plant” means all the plant species, especially those cultivated by humans, in particular food plant, which provides food for human consumption (cereals, cultures fodder crops, vegetable, fruit, vine, etc.), and/or for the supply of wood of all destinations (heating, construction of residences, furniture, etc.) and/or decoration. For example, a plant includes wheat and corn. The term plant also includes the part of plants, such as trunk, stem, branch, and leaves.

As used herein, the term “decontaminating” agent or “decontaminating” composition refers to an agent or a composition that neutralizes or removes any dangerous substances or organisms such as bacteria and fungi.

As used herein, the term “disinfectant” agent or “disinfectant” composition refers to an agent or a composition that cleans a surface or an inert object, particularly by killing eradicating, or eliminating micro-organisms such as a bacterium and a fungus.

As used herein, the term “effective amount” refers to a quantity of an active ingredient or of a composition which controls the bacterium and/or the fungus, or prevents and/or treat a bacterial and/or a fungal infection. It is obvious that the quantity to be administered can be adapted by the man skilled in the art according to the surface, to the inert object to be decontaminated or disinfected, or to the subject to be treated, to the nature of the infection etc.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

### Compounds

The present invention provides new compounds having antimicrobial and antifungal properties.

According to the invention, a compound, the salts and the stereoisomers thereof has the following formula (I):

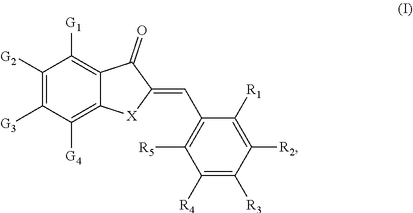

wherein:
X represents O, NH, S, or CH₂—;
G₁ represents a hydrogen or a (C₁-C₆)alkyloxy;
G₂ represents a radical selected in the group consisting of:
   a hydrogen,
   a (C₁-C₆)alkyl optionally substituted by a —NR₆R₇ group with R₆ and R₇ being independently a hydrogen or a —COR₈ with R₈ being a (C₁-C₆)alkyl;
   a —NR₆R₇ group with R₆ and R₇ being independently a hydrogen, a —COR₈ with R₈ being a (C₁-C₆)alkyl, or an arginine residue;
   a carbamimidoyl-guanidine;
   a —COOR₉ with R₉ being a hydrogen, a (C₁-C₁₂)alkyl, a geranyl, a farnesyl or a citronnellyl;
G₃ represents a radical selected in the group consisting of:
   a hydrogen,
   a hydroxy,
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
   with the proviso that when G₂ and G₃ represent a hydrogen, then:
R₁ and R₃ represent a benzyloxy, or
R₃ represents a —O-flavone optionally substituted by at least a (C₁-C₁₀)alkyloxy or a hydroxy, or a —CH₂-berberine; and
   with the proviso that said compound of formula (I) is not a compound selected in the group consisting of:
(Z)-N-(2-(4-methoxybenzylidene)-3-oxo-2,3-dihydrobenzofuran-5-yl)acetamide;
(Z)-N-(3-oxo-2-(3,4,5-trimethoxybenzylidene)-2,3-dihydrobenzofuran-5-yl)acetamide; and
(Z)-N-(2-benzylidene-3-oxo-2,3-dihydrobenzofuran-5-yl)acetamide.

In a particular embodiment, said compound of formula (I) is no longer a compound selected in the group consisting of:
AD-1-61: (2Z)-2-(3-methoxybenzylidene)-3-oxo-2,3-dihydro-1-benzofuran-5-carboxylic acid; and
AD-1-62: (2Z)-2-[4-(dimethylamino)benzylidene]-3-oxo-2,3-dihydro-1-benzofuran-5-carboxylic acid.

In a further particular embodiment, a compound, the salts and the stereoisomers thereof has the following formula (I):

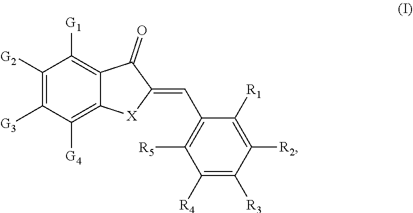

wherein:
X represents O, NH, S, or CH₂—;
G₁ represents a hydrogen or a (C₁-C₆)alkyloxy;
G₂ represents a radical selected in the group consisting of:
   a hydrogen,
   a (C₁-C₆)alkyl optionally substituted by a —NR₆R₇ group with R₆ and R₇ being independently a hydrogen or a —COR₈ with R₈ being a (C₁-C₆)alkyl;
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₃ represents a radical selected in the group consisting of:
   a hydrogen,
   a hydroxy,
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
   with the proviso that when G₂ and G₃ represent a hydrogen, then:
R₁ and R₃ represent a benzyloxy, or
R₃ represents a —O-flavone optionally substituted by at least a (C₁-C₁₀)alkyloxy or a hydroxy, or a —CH₂-berberine; and
   with the proviso that said compound of formula (I) is not a compound selected in the group consisting of:
(Z)-N-(2-(4-methoxybenzylidene)-3-oxo-2,3-dihydrobenzofuran-5-yl)acetamide;
(Z)-N-(3-oxo-2-(3,4,5-trimethoxybenzylidene)-2,3-dihydrobenzofuran-5-yl)acetamide; and
(Z)-N-(2-benzylidene-3-oxo-2,3-dihydrobenzofuran-5-yl)acetamide.

In a preferred embodiment, X represents O.

The compounds of formula (I) according to the invention can be further classified in two categories, namely “monomer” compounds or “dimer” compounds according to the definition of R₃.

### Monomer Compounds

The “monomer” compounds are compounds of formula (I) in which, R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl. Accordingly, a particular aspect of the invention is such that R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl. Preferably, R₃ represents a hydrogen, a methoxy, an isopropyloxy, a —COOH, a phenyloxy, a benzyloxy, a —O-(CH₂)₅—COOH, or a —N(CH₃)₂. More preferably, R₃ represents a hydrogen, a methoxy, an isopropyloxy, or a benzyloxy.

In this particular aspect, G₁ represents a hydrogen or a (C₁-C₆)alkyloxy, preferably a methoxy. More preferably, G₁ represents a hydrogen.

In this particular aspect, G₂ represents a radical selected in the group consisting of:
a hydrogen,
a (C₁-C₆)alkyl optionally substituted by a —NR₆R₇ group with R₆ and R₇ being independently a hydrogen or a —COR₈ with R₈ being a (C₁-C₆)alkyl;
a —NR₆R₇ group with R₆ and R₇ being independently a hydrogen, a —COR₈ with R₈ being a (C₁-C₆)alkyl, or an arginine residue; and
a carbamimidoyl-guanidine.

In a preferred embodiment, G₂ represents a radical selected in the group consisting of:
a hydrogen,
a (C₁-C₆)alkyl, preferably a methyl, and
a —NR₆R₇ group with R₆ and R₇ being independently a hydrogen, or a —COR₈ with R₈ being a (C₁-C₆)alkyl, preferably a methyl.

In a more preferred embodiment, G₂ represents a radical selected in the group consisting of:
a hydrogen, and
a —NR₆R₇ group with R₆ and R₇ being independently a hydrogen, or a —COR₈ with R₈ being a (C₁-C₆)alkyl, preferably a methyl.

In an even more preferred embodiment, G₂ represents a hydrogen or a —NHCOCH₃ group.

In this particular aspect, G₂ also represents a —COOR₉ with R₉ being a hydrogen, a (C₁-C₁₂)alkyl, a (C₁-C₆)alkyl, preferably a methyl or an ethyl, a geranyl, a farnesyl, or a citronnellyl.

In a preferred embodiment, G₂ represents a —COOH.

In this particular aspect, G₃ represents a radical selected in the group consisting of:
a hydrogen,
a hydroxy,
a —NR₆R₇ group with R₆ and R₇ being independently a hydrogen, a —COR₈ with R₈ being a (C₁-C₆)alkyl, or an arginine residue; and
a carbamimidoyl-guanidine.

In a preferred embodiment, G₃ represents a radical selected in the group consisting of:
a hydrogen,
a hydroxy,
a —NR₆R₇ group with R₆ and R₇ being independently a hydrogen, a —COR₈ with R₈ being a (C₁-C₆)alkyl, preferably a methyl.

In a more preferred embodiment, G₃ represents a radical selected in the group consisting of:
a hydrogen, and
a —NR₆R₇ group with R₆ and R₇ being independently a hydrogen, or a —COR₈ with R₈ being a (C₁-C₆)alkyl, preferably a methyl.

In an even more preferred embodiment, G₃ represents a hydrogen, a hydroxy, or an amino group, advantageously a hydrogen or an amino group.

A preferred embodiment is such that:
G₂ represents a hydrogen, or a —NR₆R₇ group with R₆ and R₇ being independently a hydrogen or a —COR₈ with R₈ being a (C₁-C₆)alkyl, preferably a methyl; and
G₃ represents a hydrogen, or a —NR₆R₇ group with R₆ and R₇ being independently a hydrogen or a —COR₈ with R₈ being a (C₁-C₆)alkyl, preferably a methyl;
   with the proviso that when one of G₂ and G₃ represents a —NR₆R₇ group with R₆ and R₇ being independently a hydrogen or a —COR₈ with R₈ being a (C₁-C₆)alkyl, preferably a methyl, then the other represents a hydrogen.

A more preferred embodiment, is such that one of G₂ and G₃ is a —NHCOR₈ group with R₈ being a (C₁-C₆)alkyl, preferably a methyl, and the other hydrogen.

A further more preferred embodiment, is such that one of G₂ and G₃ is an amino group, and the other hydrogen.

In this particular aspect, G₄ represents a hydrogen, a nitro, or a (C₁-C₆)alkyl, preferably a methyl. More preferably, G₄ is a hydrogen.

In this particular aspect, R₁ and R₅ represent a hydrogen, a halogen, a (C₁-C₆)alkyloxy, a benzyloxy, or a phenyloxy. In a preferred embodiment, R₁ and R₅ represent a hydrogen, a (C₁-C₆)alkyloxy, a benzyloxy, or a phenyloxy. In a further preferred embodiment, R₁ and R₅ represent a hydrogen, a halogen, preferably a fluorine, a (C₁-C₆)alkyloxy, preferably a methoxy, or a benzyloxy. In a specific embodiment, R₁ represents a hydrogen, a halogen, preferably a fluorine, a (C₁-C₆)alkyloxy, preferably a methoxy, or a benzyloxy. More specifically, R₁ represents a hydrogen or a benzyloxy. In a preferred embodiment, R₅ represents a hydrogen.

A more preferred embodiment, is such that both G₂ and G₃ are a hydrogen, and R₁ and R₃ or R₃ and R₅ are a benzyloxy.

In this particular embodiment, R₂ and R₄ represent a hydrogen, a halogen, a (C₁-C₆)alkyloxy, a phenyloxy, or a benzyloxy. Preferably, R₂ and R₄ represent a hydrogen, a halogen, preferably a fluorine, a (C₁-C₆)alkyloxy, preferably a methoxy or an isopropyloxy, a phenyloxy, or a benzyloxy. In a preferred embodiment, R₂ represents a hydrogen, a halogen, preferably a fluorine, a (C₁-C₆)alkyloxy, preferably a methoxy or an isopropyloxy, a phenyloxy, or a benzyloxy. More preferably, R₂ represents a hydrogen or a benzyloxy. In a preferred embodiment, R₄ represents a hydrogen or a methoxy, preferably a hydrogen.

In a further particular embodiment, R₃ and R₄ represent a benzyloxy or R₂ and R₃ represent a benzyloxy.

In this particular aspect, a compound of formula (I) is selected in the group consisting of:
A1: (Z)-N-(2-(2′-methoxybenzylidene)-3-oxo-2,3-dihydrobenzofuran-5-yl)acetamide;
A2: (Z)-N-(2-(3-methoxybenzylidene)-3-oxo-2,3-dihydrobenzofuran-5-yl)acetamide;
A7: (Z)-N-(2-(3-(benzyloxy)benzylidene)-3-oxo-2,3-dihydrobenzofuran-5-yl)acetamide;
A9: (Z)-5-amino-2-(3-(benzyloxy)benzylidene)benzofuran-3(2H)-one;
A12: (Z)-N-(3-oxo-2-(4-phenoxybenzylidene)-2,3-dihydrobenzofuran-5-yl)acetamide;
B1: (Z)-5-amino-2-(3-phenoxybenzylidene)benzofuran-3(2H)-one;
B4: (Z)-N-(2-(3-isopropoxybenzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
B10: (Z)-N-(2-(3-fluorobenzylidene)-3-oxo-2,3-dihydrobenzofuran-5-yl)acetamide;
B11: (Z)-5-amino-2-(2-fluorobenzylidene)benzofuran-3(2H)-one;
C4: (Z)-4-((5-acetamido-3-oxobenzofuran-2(3H)-ylidene)methyl)benzoic acid;
C8: (Z)-N-(7-nitro-3-oxo-2-(3-phenoxybenzylidene)-2,3-dihydrobenzofuran-5-yl)acetamide;
C10: (Z)-2-benzylidene-6-hydroxy-4-methoxy-5,7-dimethylbenzofuran-3(2H)-one;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
D2: (Z)-5-amino-2-(3,4,5-trimethoxybenzylidene)benzofuran-3(2H)-one; and
D3: (Z)-N-(3-oxo-2-(2,4,5-trimethoxybenzylidene)-2,3-dihydrobenzofuran-5-yl)acetamide.

Preferably said compound is selected in the group consisting of:
A7: (Z)-N-(2-(3-(benzyloxy)benzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
AD-1-44: (Z)-N-(2-(3,4-bis(benzyloxy)benzylidene)-3-oxo-2,3-dihydrobenzofuran-5-yl)acetamide;
MR1065 : (Z)-2-(3,4-bis(benzyloxy)benzylidene)-3-oxo-2,3-dihydrobenzofuran-5-carboxylic acid;
MR1076: (Z)-6-(4-((3-oxobenzofuran-2(3H)-ylidene)methyl)phenoxy)hexanoic acid;
MR1120: (Z)-ethyl 2-(3,4-bis(benzyloxy)benzylidene)-3-oxo-2,3-dihydrobenzofuran-5-carboxylate; and
MR1121: (Z)-decyl 2-(3,4-bis(benzyloxy)benzylidene)-3-oxo-2,3-dihydrobenzofuran-5-carboxylate.

### Dimer Compounds

The “dimer” compounds are compounds of formula (I) in which, R₃ represents a —O-flavone optionally substituted by at least a (C₁-C₁₀)alkyloxy or a hydroxy, or a —CH₂-berberine.

Accordingly, a particular aspect of the invention is such that R₃ represents a —O-flavone optionally substituted by at least a (C₁-C₁₀)alkyloxy or a hydroxy, or a —CH₂-berberine. In a preferred embodiment, R₃ is a —O-flavone optionally substituted by at least a (C₁-C₁₀)alkyloxy or a hydroxy. Preferably, R₃ is a —O-flavone. In a further preferred embodiment, R₃ is a —CH₂-berberine.

In this particular aspect, G₁ represents a hydrogen or a (C₁-C₆)alkyloxy. Preferably, G₁ represents a hydrogen.

In this particular aspect, G₂ represents a radical selected in the group consisting of:
a hydrogen,
a (C₁-C₆)alkyl optionally substituted by a —NR₆R₇ group with R₆ and R₇ being independently a hydrogen or a —COR₈ with R₈ being a (C₁-C₆)alkyl;
a —NR₆R₇ group with R₆ and R₇ being independently a hydrogen, a —COR₈ with R₈ being a (C₁-C₆)alkyl, or an arginine residue; and
a carbamimidoyl-guanidine.

Preferably, G₂ represents a hydrogen.

In this particular aspect, G₃ represents a radical selected in the group consisting of:
a hydrogen,
a hydroxy,
a —NR₆R₇ group with R₆ and R₇ being independently a hydrogen, a —COR₈ with R₈ being a (C₁-C₆)alkyl, or an arginine residue; and
a carbamimidoyl-guanidine.

Preferably, G₃ represents a hydrogen.

A preferred embodiment, is such that both G₂ and G₃ are a hydrogen, and R₃ represents a —O-flavone optionally substituted by at least a (C₁-C₁₀)alkyloxy or a hydroxy, or a —CH₂-berberine.

In this particular aspect, G₄ represents a hydrogen, a nitro, or a (C₁-C₆)alkyl, preferably a methyl. Preferably, G₄ represents a hydrogen.

In this particular aspect, R₁ and R₅ represent a hydrogen, a halogen, a (C₁-C₆)alkyloxy, a benzyloxy, or a phenyloxy. Preferably, R₁ and R₅ represent a hydrogen.

In this particular embodiment, R₂ and R₄ represent a hydrogen, a halogen, a (C₁-C₆)alkyloxy, a phenyloxy, or a benzyloxy. Preferably, R₂ and R₄ represent a hydrogen.

In this particular aspect, a compound of formula (I) is selected in the group consisting of:
E5: (Z)-6-(4-((3-oxobenzofuran-2(3H)-ylidene)methyl)phenoxy)-2-phenyl-4H-chromen-4-one; and
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide.

### Applications

As illustrated by the Examples, the inventors have demonstrated an antimicrobial, in particular antibacterial and an antifungal, effect for the compounds of formula (I). Accordingly, the compounds as defined herein, particularly including monomer and dimer compounds, can be useful for controlling a bacterium and/or a fungus.

Thus, the present invention further relates to a use of a compound of formula (I), the salts and the stereoisomers thereof:

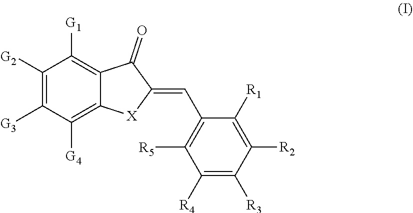

wherein:
X represents O, NH, S, or CH₂—;
G₁ represents a hydrogen or a (C₁-C₆)alkyloxy;
G₂ represents a radical selected in the group consisting of:
   a hydrogen,
   a (C₁-C₆)alkyl optionally substituted by a —NR₆R₇ group with R₆ and R₇ being independently a hydrogen or a —COR₈ with R₈ being a (C₁-C₆)alkyl;
   a —NR₆R₇ group with R₆ and R₇ being independently a hydrogen, a —COR₈ with R₈ being a (C₁-C₆)alkyl, or an arginine residue;
   a carbamimidoyl-guanidine; and
   a —COOR₉ with R₉ being a hydrogen, a (C₁-C₁₂)alkyl, a geranyl, a farnesyl or a citronnellyl;
G₃ represents a radical selected in the group consisting of:
   a hydrogen,
   a hydroxy,
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
   with the proviso that when G₂ and G₃ represent a hydrogen, then:
R₁ and R₃ represent a benzyloxy, or
R₃ represents a —O-flavone optionally substituted by at least a (C₁-C₁₀)alkyloxy or a hydroxy, or a —CH₂-berberine;
   or a compound D10: (Z)-2-(4-methoxybenzylidene)benzofuran-3(2H)-one;
   for controlling a bacterium and/or a fungus.

In a particular embodiment, invention relates to a use of a compound of formula (I), the salts and the stereoisomers thereof:

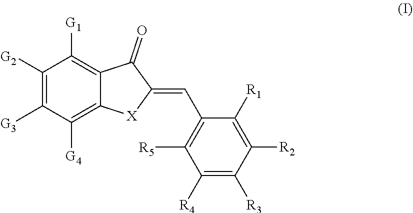

wherein:
X represents O, NH, S, or CH₂—;
G₁ represents a hydrogen or a (C₁-C₆)alkyloxy;
G₂ represents a radical selected in the group consisting of:
   a hydrogen,
   a (C₁-C₆)alkyl optionally substituted by a —NR₆R₇ group with R₆ and R₇ being independently a hydrogen or a —COR₈ with R₈ being a (C₁-C₆)alkyl;
   a —NR₆R₇ group with R₆ and R₇ being independently a hydrogen, a —COR₈ with R₈ being a (C₁-C₆)alkyl, or an arginine residue; and
   a carbamimidoyl-guanidine; and
G₃ represents a radical selected in the group consisting of:
   a hydrogen,
   a hydroxy,
   a —NR₆R₇ group with R₆ and R₇ being independently a hydrogen, a —COR₈ with R₈ being a (C₁-C₆)alkyl, or an arginine residue; and
   a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
   with the proviso that when G₂ and G₃ represent a hydrogen, then:
R₁ and R₃ represent a benzyloxy, or
R₃ represents a —O-flavone optionally substituted by at least a (C₁-C₁₀)alkyloxy or a hydroxy, or a —CH₂-berberine;
   or a compound D10: (Z)-2-(4-methoxybenzylidene)benzofuran-3(2H)-one;
   for controlling a bacterium and/or a fungus.

The present invention also relates to a use of a compound of formula (I) in which X, G₁, G₂, G₃, G₄, R₁, R₂, R₃, R₄, R₅, are such as above defined, including all the particular and preferred embodiments and aspects as above defined, or a compound D10: (Z)-2-(4-methoxybenzylidene)benzofuran-3(2H)-one for controlling a bacterium and/or a fungus.

A preferred object of the invention is a use of a compound for controlling a bacterium and/or a fungus, wherein said compound is is selected in the group consisting of:
A1: (Z)-N-(2-(2′-methoxybenzylidene)-3-oxo-2,3-dihydrobenzofuran-5-yl)acetamide;
A2: (Z)-N-(2-(3-methoxybenzylidene)-3-oxo-2,3-dihydrobenzofuran-5-yl)acetamide;
A7: (Z)-N-(2-(3-(benzyloxy)benzylidene)-3-oxo-2,3-dihydrobenzofuran-5-yl)acetamide;
A9: (Z)-5-amino-2-(3-(benzyloxy)benzylidene)benzofuran-3(2H)-one;
A12: (Z)-N-(3-oxo-2-(4-phenoxybenzylidene)-2,3-dihydrobenzofuran-5-yl)acetamide;
B1: (Z)-5-amino-2-(3-phenoxybenzylidene)benzofuran-3(2H)-one;
B4: (Z)-N-(2-(3-isopropoxybenzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
B10: (Z)-N-(2-(3-fluorobenzylidene)-3-oxo-2,3-dihydrobenzofuran-5-yl)acetamide;
B11: (Z)-5-amino-2-(2-fluorobenzylidene)benzofuran-3(2H)-one;
C4: (Z)-4-((5-acetamido-3-oxobenzofuran-2(3H)-ylidene)methyl)benzoic acid;
C8: (Z)-N-(7-nitro-3-oxo-2-(3-phenoxybenzylidene)-2,3-dihydrobenzofuran-5-yl)acetamide;
C10: (Z)-2-benzylidene-6-hydroxy-4-methoxy-5,7-dimethylbenzofuran-3(2H)-one;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
D1: (Z)-N-(3-oxo-2-(3,4,5-trimethoxybenzylidene)-2,3-dihydrobenzofuran-5-yl)acetamide;
D2: (Z)-5-amino-2-(3,4,5-trimethoxybenzylidene)benzofuran-3(2H)-one;
D3: (Z)-N-(3-oxo-2-(2,4,5-trimethoxybenzylidene)-2,3-dihydrobenzofuran-5-yl)acetamide;
D8: (Z)-N-(2-benzylidene-3-oxo-2,3-dihydrobenzofuran-5-yl)acetamide;
D10: (Z)-2-(4-methoxybenzylidene)benzofuran-3(2H)-one;
E5: (Z)-6-(4-((3-oxobenzofuran-2(3H)-ylidene)methyl)phenoxy)-2-phenyl-4H-chromen-4-one;
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide;
AD-1-44: (Z)-N-(2-(3,4-bis(benzyloxy)benzylidene)-3-oxo-2,3-dihydrobenzofuran-5-yl)acetamide;
AD-1-61: (2Z)-2-(3-methoxybenzylidene)-3-oxo-2,3-dihydro-1-benzofuran-5-carboxylic acid;
AD-1-62: (2Z)-2-[4-(dimethylamino)benzylidene]-3-oxo-2,3-dihydro-1-benzofuran-5-carboxylic acid;
MR1065: (Z)-2-(3,4-bis(benzyloxy)benzylidene)-3-oxo-2,3-dihydrobenzofuran-5-carboxylic acid;
MR1076: (Z)-6-(4-((3-oxobenzofuran-2(3H)-ylidene)methyl)phenoxy)hexanoic acid;
MR1120: (Z)-ethyl 2-(3,4-bis(benzyloxy)benzylidene)-3-oxo-2,3-dihydrobenzofuran-5-carboxylate; and
MR1121: (Z)-decyl 2-(3,4-bis(benzyloxy)benzylidene)-3-oxo-2,3-dihydrobenzofuran-5-carboxylate.

A more preferred object of the invention is a use of a compound for controlling a bacterium and/or a fungus, wherein said compound is is selected in the group consisting of:
MR1065: (Z)-2-(3,4-bis(benzyloxy)benzylidene)-3-oxo-2,3-dihydrobenzofuran-5-carboxylic acid;
A7: (Z)-N-(2-(3-(benzyloxy)benzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
E5: (Z)-6-(4-((3-oxobenzofuran-2(3H)-ylidene)methyl)phenoxy)-2-phenyl-4H-chromen-4-one; and
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11. 8.0.0^{2,10}.0^{4,8}.0^{15,20}]henicosa-1(21),2,4(8),9,13,15(20),16,18-octaen-13-ylium bromide.

Particularly, the bacterium is a Gram⁺ bacterium, a Gram⁻ bacterium, or a Mycobacterium.

In a particular embodiment, the bacterium is a Gram⁺ bacterium.

Preferably, the Gram⁺ bacterium is selected in the group consisting of: Bacillus cereus, Bacillus subtilis, Clostridium botulinum, Clostridium coccoides, Clostridium difficile, Clostridium perfringens, Clostridium propionicum, Enterococcus faecalis, Lactococcus lactis, Listeria monocytogenes, Micrococcus luteus, Propionibacterium acnes, Staphylococcus aureus, Streptococcus pyogenes, and Streptomyces roietensis.

In a further aspect, the Gram⁺ bacterium is a Gram⁺ bacterium resistant to any antibiotic. As an example of antibiotic resistant Gram⁺ bacterium, it may be cited, without limitation, bacteria belonging to genera Bacillus, Enterococcus, Staphylococcus, Clostridium, Listeria, and Streptococcus. Preferably, the antibiotic resistant Gram⁺ bacterium is selected in the group consisting of: B. subtilis Nisin R, and S. aureus Methicillin R.

In a further particular embodiment, the bacterium is a Gram⁻ bacterium.

Preferably, the Gram⁻ bacterium is selected in the group consisting of: Acinetobacter baumannii, Bacteroides thetaioataomicron, Burkholderia cepacia, Burkholderia thailandensis, Burkholderia mallei, Burkholderia pseudomallei, Citrobacter farmer, Citrobacter rodentium, Escherichia coli, E. coli EHEC, Helicobacter pylori, Klebsiella pneumoniae, Klebsiella variicola, Pseudomonas aeruginosa, Pseudomonas syringae, Salmonella enterica, Shigella flexneri, and Vibrio alginolyticus.

In a further aspect, the Gram⁻ bacterium is a Gram⁻ bacterium resistant to any antibiotic. As an example of antibiotic resistant Gram⁻ bacterium, it may be cited, without limitation, bacteria belonging to the genera Klebsiella, Pseudomonas, Helicobacter, Enterobacter, Vibrio, Burkholderia, Neisseria, Agrobacterium. Preferably, the antibiotic resistant Gram⁻ bacterium is Burkholderia cepacia, Burkholderia thai, Burkholderia mallei and Burkholderia pseudomallei. Particularly, such Gram⁻ Burkholderia bacteria are naturally resistant to antibiotics such as cephalosporins, penicillin, macrolides, aminosides.

In a further particular embodiment, the bacterium is a Mycobacterium. As an example of Mycobacterium, it may be cited without limitation M. tuberculosis, M. avium, M. avium paratuberculosis, M. marinum, M. abscessus, M. ulcerans, M. leprae or M. smegmatis. Preferably, the Mycobacterium is M. smegmatis.

Particularly, the fungus is selected in the group consisting of: Candida albicans, Metarhizium anisopliae, Beauveria feline, Aspergillus flavus, Fusarium graminearum, Fusarium verticillioides, Penicillium verrucosum, Fusarium oxysporum, Aspergillus niger, Stachybotrys chartarum, Aspergillus ochraceus, Microdochium bolleyi, Microdochium majus, and Microdochium nivale.

The invention also concerns a use of a compound of formula (I) as defined herein for reducing, eliminating, eradicating, killing, decontaminating preventing, and/or treating a bacterium and/or a fungus. The invention further concerns a use of a compound of formula (I) as defined herein for slowing down and/or stopping the development of the bacterium and/or the fungus.

The invention further concerns a method for controlling a bacterium and/or a fungus comprising applying an effective amount of a compound of formula (I) as defined herein, on a surface or an inert object. Another object of the invention is a method for reducing, eliminating, eradicating, killing, decontaminating, preventing, and/or treating a bacterium and/or a fungus or for slowing down and/or stopping the development of the bacterium and/or the fungus, comprising applying an effective amount of a compound of formula (I) as defined herein, on a surface or an inert object.

A further object of the invention is a use of a compound as defined herein as a phytoprotective and/or decontaminating and/or disinfectant agent. Preferably, the phytoprotective and/or decontaminating and/or disinfectant agent is applied on a surface or an inert object. In a particular embodiment, the phytoprotective and/or decontaminating and/or disinfectant agent is applied in combination with at least an anti-microbial and/or an anti-fungal agent.

A further object of the invention is a phytoprotective or decontaminating or disinfectant composition comprising a compound as defined herein. In a particular embodiment, said phytoprotective or decontaminating or disinfectant composition further comprises an anti-microbial and/or anti-fungal agent.

As an example of anti-microbial agent, it may be cited, without limitation, i) antibiotics used in medicine such as the one belonging to the following families: Penicillins, Tetracyclines, Cephalosporins, Quinolones, Lincomycins, Macrolides, Sulfonamides, Glycopeptides, Aminoglycosides, Carbapenems, and Antimicrobial peptides, and ii) anti-microbial agents used to treat surface such as alcohols, sodium hydroxide, bleech, detergents, and cationic detergents.

As an example of anti-fungal agent, it may be cited, without limitation, i) antifungal drugs used in medicine such as clotrimazole, econazole, miconazole, terbinafine, fluconazole, ketonazole, and amphotericin, and ii) antifungal agents used as phytosanitary treatment, such as metconazole, fludioxonil, tébuconazole, difénoconazole, prothioconazole, sedaxane and copper sulfate solution.

Particularly, the phytoprotective and/or decontaminating and/or disinfectant agent is used at a concentration between 0.1 and 100 μM, 0.5 and 50 μM, 0.5 and 30 μM, preferably 0.78 and 25 μM, more preferably 25 μM. Accordingly, the phytoprotective or decontaminating or disinfectant composition comprises a compound as defined herein in a concentration comprised between 0.1 and 100 μM, 0.5 and 50 μM, 0.5 and 30 μM, preferably 0.78 and 25 μM, more preferably 25 μM.

The phytoprotective or decontaminating or disinfectant composition as defined herein may also comprise any excipient currently used in the cleansing, phytoprotective, decontaminating, and disinfecting fields.

The invention also relates to a process for the decontamination or the disinfection of a surface or an inert object comprising a step of applying a phytoprotective or decontaminating or disinfectant composition as defined herein, on a surface or an inert object infested or suspected to be infested by a bacterium and/or a fungus.

The invention also concerns a phytosanitary process comprising a step of applying a phytoprotective composition as defined herein on a plant (or in parts of a plant) infested or suspected to be infested by a bacterium and/or a fungus. Preferably, the plant is chosen cereals, cultures fodder crops, vegetable, fruit, vine, more preferably wheat and corn.

The invention further relates to a kit comprising a compound of formula (I) as defined herein, and an anti-microbial and/or anti-fungal agent as a combined preparation for simultaneous, separate, or sequential use, in particular for controlling a bacterium and/or a fungus. In a preferred embodiment, the kit further comprises a notice or an instruction guide, particularly for controlling bacterium and/or a fungus.

In another aspect, the invention relates to a compound as defined herein for use as a medicine or a drug. In particular, the present invention also relates to a compound as defined herein for use for preventing and/or treating a bacterial infection and/or a fungal infection. The present invention also relates to a compound as defined herein for use as an antibiotic or as an antifungal medication.

The invention also relates to a pharmaceutical composition comprising a compound as defined herein, and a pharmaceutically acceptable excipient.

Another object of the invention is a pharmaceutical composition as defined herein for use for preventing and/or treating a bacterial infection and/or a fungal infection. The invention also relates to a method for treating a bacterial infection and/or a fungal infection comprising administering an effective amount a compound of formula (I) as defined herein or a pharmaceutical composition comprising such compound of formula (I) in a subject in need thereof, preferably a mammal, more preferably a human. The invention also relates to a use of a compound of formula (I) as defined for the manufacture of a pharmaceutical composition for preventing and/or treating a bacterial infection and/or a fungal infection.

As used herein, a bacterial infection is the invasion (either local or systemic, causing or not a disease) of the host (either an animal, a human or a plant) by a bacterium belonging to the Gram+, the Gram− or the Mycobacterium family as above defined.

As used herein, a fungal infection is the invasion (either local or systemic, causing or not a disease) of the host (either an animal, a human or a plant) by a fungus, preferably a fungus belonging to the yeast, the Ascomycota, the Basidiomycota or the monogeneric family.

Further aspects and advantages of the present invention will be described in the following examples, which should be regarded as illustrative and not limiting.

### EXAMPLES

### Example A: Chemistry

### 1. Preparation of the Compounds

Route 1: Preparation of the 5-acetamido chalcone derivatives have been done by the Claisen-Schmidt condensation of substituted benzaldehydes and 2-hydroxy-5-acetamidoacetophenone (2) has drawn below.

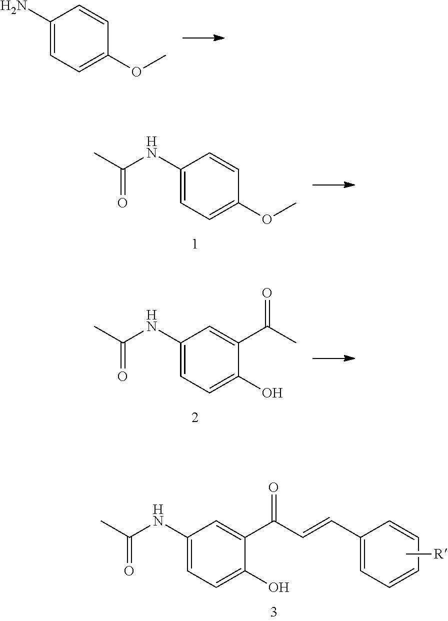

To a stirring suspension of p-anisidine (6.036 g, 49 mmol) in DCM (20 mL), acetic anhydride (5 mL, 53 mmol) was added dropwise over a period of 1 h. The reaction was stirred for 1 h, then poured into hexane (60 mL) and stirred for further 1 h. The solid formed was collected by filtration and washed with hexane to afford the title compound 1 (7.717 g, 95%, mp: 132° C.) as a pale grey solid. HRMS: calcd for C9H11NO2 165.0790 Found, 165.0789. LRMS (EI): 165 (M+, 71%), 108 ([NH2C6H4O]+, 100). 1H NMR (400 MHz, CDCl3) δ 2.13 (s, 3H) 3.78 (s, 3H) 6.83 (d, 2H, 9 Hz) 7.38 (d, 2H, 9 Hz). 13C NMR (100 MHz, CDCl3) δ 24.66, 55.85, 114.49, 122.37, 131.41, 156.82, 168.79.

To a stirring suspension of 1 (32 mmol) and acetyl chloride (93 mmol) in DCM, aluminium trichloride (AlCl₃, 109 mmol) was added in portions over 90 min. The reaction was then heated to reflux for 4.5 h and cooled overnight. The mixture was poured onto ice, then extracted with DCM (5×), dried (MgSO4) and concentrated in vacuo to give N-(3-acetyl-4-hydroxy-phenyl)-acetamide (2). The title compound was crystallized from diethyl ether as a pale green solid (5.336 g, 87%, mp: 125 163° C.). HRMS: calcd for C10H11NO3 193.0739 Found, 193.0740. Anal. Calcd. for C10H11NO3: C, 62.17; H, 5.74; N, 7.25. Found: C, 62.11; H, 5.81; N, 7.12. LRMS (EI): 193 (M+⋅, 100%). 1H NMR (300 MHz, CDCl3) δ 2.16 (s, 3H) 2.61 (s, 3H) 6.92 (d, 1H, J=9 Hz) 7.33 (dd, 1H, J=2.6 and 9 Hz) 8.16 (d, 1H, J=2.6 Hz) 12.09 (s, 1H, OH). 13C NMR (75 MHz, CDCl3) δ 24.71, 27.16, 119.08, 119.60, 122.94, 127.42, 129.58, 159.62, 168.86, 204.84.

Preparation of Chalcone 3 (Claisen-Schmidt Condensation).

A mixture of 2 (10 mmol), corresponding aldehyde (10 mmol) and LiOH·H₂O (70 mmol) in MeOH (20 mL) was submitted to MWI in a CEM Apparatus (open vessel, 300 watts, at 80° C.) for 2 min+20 min. The resulting solution was allowed to cool to r.t.; the solvent was removed under vacuum and the residue was poured into 50 mL HCl 1N. The precipitate obtained was then filtered off, washed with excess water, dried and used without further purification.

Aurone Synthesis:

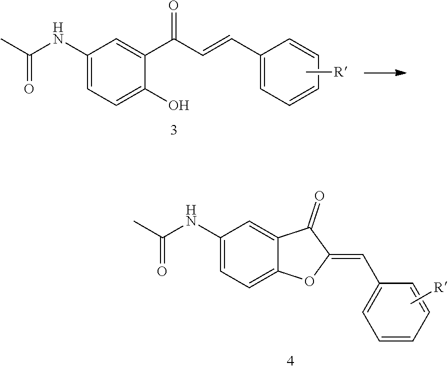

To a solution of pyridine (10 mL) in equimolar amount of mercuric diacetate (Hg(OAc)₂ 10 mmoles) and corresponding chalcone (3, 10 mmoles) was added and the solution refluxed for 1 h. The reaction was then cooled to r.t., poured onto ice-cold water and neutralized with HCl 1N. The precipitate thus obtained was filtered and crystallized with ethylacetate to give the desired aurones 4. Labelling in this study (A1, A2, A7, A12, B4, B5, B10, C4, C8, D1, D3, D8).

C10 was obtained by this synthetic procedure starting with DMC (2′,4′-dihydroxy-6′-methoxy-3′,5′-dimethylchalcone, European Food Research and Technology, 2012, 235, 1133-1139) extracted from Cleistocalix operculatus buds.

C12, D10, aurones have also obtain by this procedure starting with the corresponding chalcone.

Deprotection of acetamido chalcones (A1-2, A7, A12, B4, B5, B10, C4, C8, D1, D3, D8) in amino derivatives.

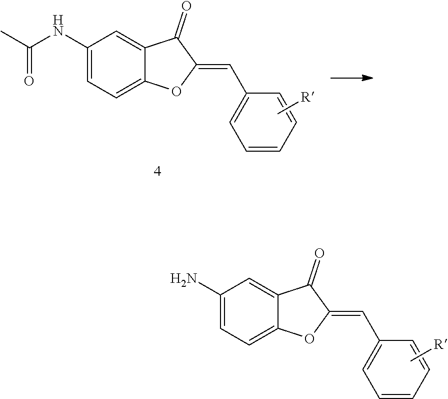

10 mmol of acetamido chalcones were added to a mixture of EtOH (20 mL) and conc. H2SO4 (5 mL). The solution was refluxed for 2 h. Upon cooling, the solvent was removed under vacuum and the residue obtained was poured onto iced water (100 mL). The resulting solution was neutralized with NH4OH 16% until pH=7. The precipitate formed was collected by filtration and washed with excess cold water. The resulting amino derivative (A9, B1, B11, C1, D2,) was used without further purification.

Route 2: Synthesis of 6-acetamido aurone was developed with the corresponding benzofuranone condensation.

Chloroacetyl chloride (6.52 g, 58.2 mmol) was added, followed within 15 min by 4 g (24.2 mmol) of N-(3 or 4-methoxyphenyl)-acetamide, to a solution of 12.92 g (98.0 mmol) of AlCl₃ in 20 mL of 1,2-dichloroethane at 0° C. under N₂. The mixture was stirred for 30 min at 0° C., slowly warmed to room temperature, and further stirred for 24 h. The brown mixture was added to 200 mL of ice-water and 200 mL of AcOEt. After the mixture had been vigorously stirred. The resulting precipitate was filtered off and dried under vacuum.

MR1060: N-(3-(2-chloroacetyl)-4-hydroxyphenyl)acetamide

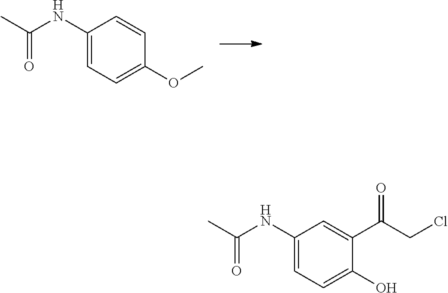

Yield: 83%; ¹H NMR (300 MHz, DMSO-d₆): δ 10.85 (s, 1H, NH), 9.90 (s, 1H, OH), 7.92 (d, 1H, J=1.9 Hz, C—H₂), 7.69-7.66 (dd, 1H, J=1.9; 8.5 Hz, C—H₆), 6.96-6.93 (d, 1H, J=8.5 Hz, C—H₇), 5.02 (s, 2H, CH₂), 2.00 (s, 3H, CH₃). ¹³C NMR (75 Mhz, DMSO-d₆): δ 192.71 (CO), 168.09 (CO), 154.73 (C-4), 131.41 (C-1), 127.32 (C-6), 120.72 (C-3), 120.33 (C-2), 117.6 (C-5), 50.31 (CH2Cl), 23.73 (CH3). Elemental analysis calcd (%) for C₁₀H₁₀ClNO₃: C, 52.76; H, 4.43; N, 6.15; found C, 52.79; H, 4.46; N, 6.11. m/z: 227,03492 (100.0%).

MR1057: N-(4-(2-chloroacetyl)-3-hydroxyphenyl)acetamide

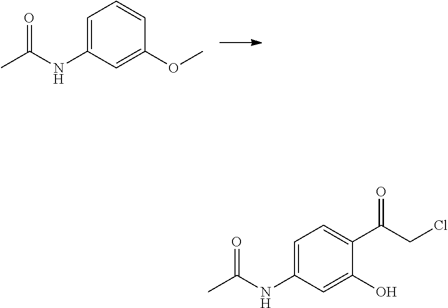

Yield: 81%; ¹H NMR (300 MHz, DMSO-d₆): δ 11.39 (s, 1H, NH), 10.25 (s, 1H, OH), 7.77-7.75 (d, 1H, J=8.8 Hz, C—H₂), 7.47 (d, 1H, J=1.8 Hz, C—H₂), 7.03-7.01 (dd, 1H, J=1.9; 8.8 Hz, C—H₆), 5.02 (s, 2H, CH₂), 2.07 (s, 3H, CH₃). ¹³C NMR (75 Mhz, DMSO-d₆): δ 192.74 (CO), 169.22 (CO), 161.11 (C-3), 146.01 (C-1), 131.45 (C-5), 114.89 (C-4), 110.23 (C-6), 105.9 (C-2), 48.68 (CH2), 24.23 (CH3). Elemental analysis calcd (%) for C₁₀H₁₀ClNO₃: C, 52.76; H, 4.43; N, 6.15; found C, 52.69; H, 4.50; N, 6.10. m/z: 227,03492 (100.0%).

MR1058: N-(3-oxo-2,3-dihydrobenzofuran-6-yl)acetamide

The resulting solid was introduced into 30 mL of EtOH; 2.64 g (32.2 mmol) of AcONa was added, and then the mixture was heated under reflux for 24 h. EtOH was removed under reduced pressure, and the mixture solid was purified by silica gel chromatography using a 1:2 (v/v) petroleum ether/AcOEt mixture as the eluent. 3 as a yellow solid (1.47 g, 32%). ¹H NMR (400 MHz, DMSO-d₆): δ 10.45 (s, 1H), 7.71 (d, 1H, J=1.4 Hz), 7.56 (d, 1H, J=8.4 Hz), 7.14 (dd, 1H, J₁=8.5 Hz, J₂=1.6 Hz), 4.75 (s, 2H), 2.11 (s, 3H). ¹³C NMR (100 MHz, DMSO-d₆): δ 197.5, 174.5, 169.4, 147.9, 124.2, 115.6, 113.4, 101.5, 75.2, 24.3. TOFEI calcd for [C₁₀H₉NO₃] m/z 191.0582, found m/z 191.0534.

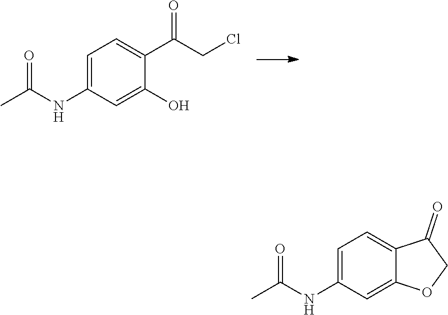

MR1061: N-(3-oxo-2,3-dihydrobenzofuran-5-yl)acetamide

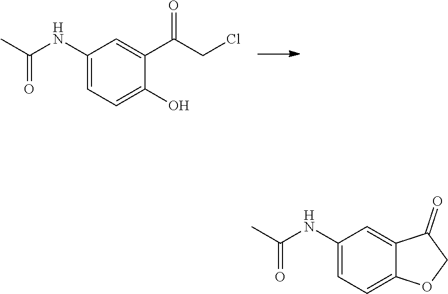

¹H NMR (300 MHz, DMSO-d₆): δ 10.09 (s, 1H, NH), 7.95 (d, 1H, J=2.2 Hz, C—H4), 7.75-7.72 (dd, 1H, J=2.4; 8.9 Hz, C—H₆), 7.25-7.22 (d, 1H, J=8.99 Hz, C—H₇), 4.78 (s, 2H, CH₂), 2.04 (s, 3H, CH₃). ¹³C NMR (75 Mhz, DMSO-d₆): δ 199.78 (C-3), 169.3 (C-8), 168.37 (CO), 133.89 (C-5), 129.83 (C-6), 120.67 (C-9), 113.69 (C-4), 112.43 (C-7), 75.25 (C-2), 23.84 (CH3). Elemental analysis calcd (%) for C₁₀H₉NO₃: C, 62.82; H, 4.74; N, 7.33; found C, 62.78; H, 4.76; N, 7.29. m/z: 191,05824 (100.0%).

MR1058 was condensed with the corresponding aldehyde in ethanol/KOH (10%) mixture and/or Choline Chloride/Urea deep eutectic solvent to give G4 was deprotected as described to give C11.

Dimer Preparation:

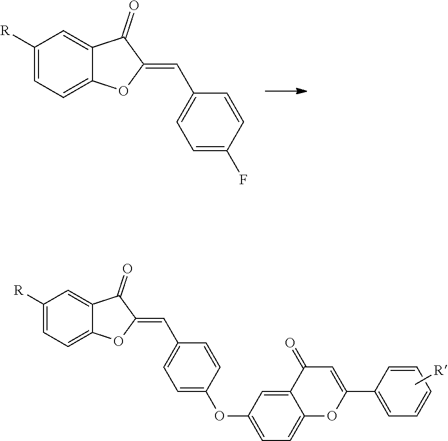

E5: 4′-fluoroaurone (D5) with 6-hydroxyflavone (CAS Number 6665-83-4)

4′-FluoroAurone (10 mmoles), 6-hydroxyFlavone (10 mmoles) and K₂CO₃ (20 mmoles, 2 eq) were mixed in Dimethylacetamide (10 mL) and heated at 130° C. for 12 h. The cooled solution was then poured into ice-water/HCl 1N until a precipitate appeared. The solution was filtered off, dried and washed with diethyl ether to give the desired dimer.

F2: Aurone-Berberine

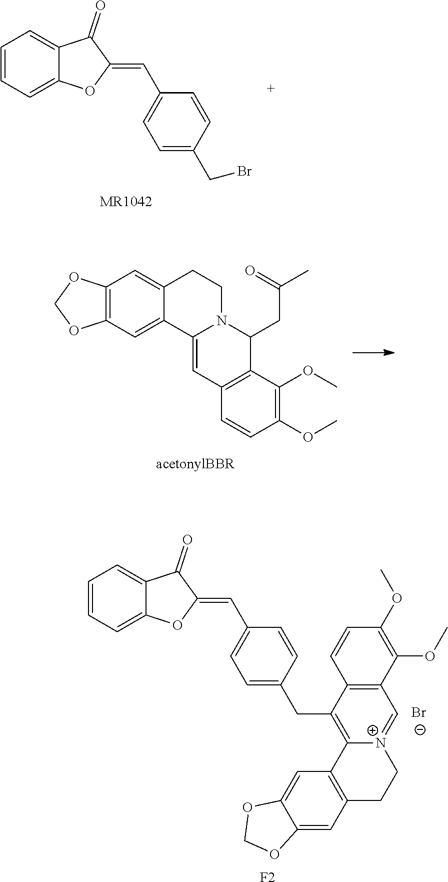

Berberine chloride (3.71 g, 10 mmol) was dissolved in 5N NaOH (20 mL) under stirring at r.t. Acetone (5 mL) was added dropwise at that temperature and stirred for 1 h, precipitation occurred during that time and the reaction mixture was filtered and washed with 80% MeOH to give the desired acetonylberberberine acetonyBBR (3.34 g, 85%). acetonylBBR (1 g, 2.5 mmol) was used without further purification, dissolved in acetonitrile, NaI (0.5 g, 3.3 mmol) was added MR1042 (3 mmol) at 80° C. for 4 h. The reaction mixture was concentrated under vaccuo and chromatographed on silica gel (CH₂CL₂/CH₃OH, 90/10 v/v) to give F2.

Preparation of the 5-carboxylic acid chalcone derivatives have been done by the Claisen-Schmidt condensation of substituted benzaldehydes and 3-acetyl-4-hydroxybenzoic acid (2) has drawn below.

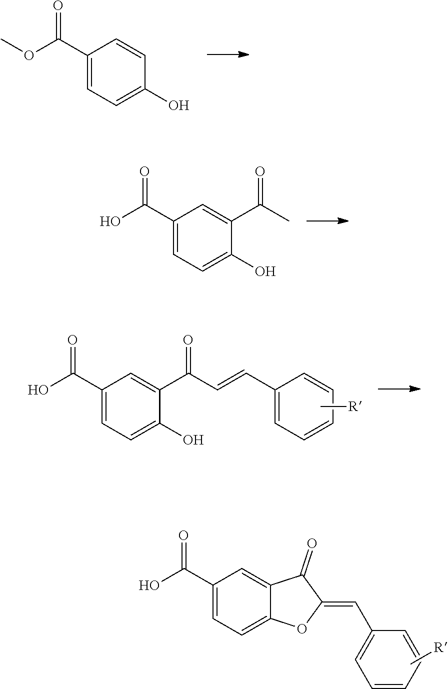

Cyclization of the corresponding chalcone to aurone (AD1-61, AD1-66 and MR1065) was done in pyridine with 1 eq of mercuric acetate at 110° C. for 1 h.

MR1120 and MR1121 are obtained starting from MR1065.

Esterification of MR1065 was obtained in EtOH at reflux with catalytic amount of H₂SO₄ for MR1120. Reflux a solution of benzoic acid (8 mmol), octan-1-ol (8 mmol) and base (4 mmol) in xylene (8 mL) for 12 h with removing water by Dean-Stark apparatus for MR1121.

### 2. Compounds

All reagents were weighed and handled in air at room temperature. ¹H NMR spectra were measured on a Brucker AC 300 and AC 400(300 and 400 MHz) spectrometer. Data were reported as follows: chemical shifts in ppm referenced to the internal solvent signal (peak at 7.26 ppm in the case of CDCl₃; peak at 2.49 in the case of DMSO-d₆), multiplicity (s=singlet, d=doublet, t=triplet, q=quartet, dd=double-doublet, m=multiplet, br=broad), coupling constants (Hz), and assignment. ¹³C NMR spectra were measured on a Brucker AC 300 and 400 (75 and 100 MHz) spectrometer with complete proton decoupling. Chemical shifts were reported in ppm from the internal solvent signal (peak at 77 ppm in the case of CDCl₃, and 39.5 for DMSO-d₆).

A1: (Z)-N-(2-(2′-methoxybenzylidene)-3-oxo-2,3-dihydrobenzofuran-5-yl)acetamide

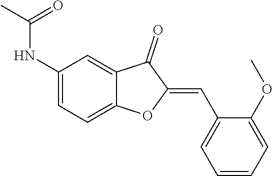 [SMILES: COc1ccccc1/C=C1\Oc2ccc(NC(C)=O)cc2C1=O]

Yield: 83%; ¹H NMR (300 MHz, DMSO-d₆): δ 10.15 (s, 1H, NH), 8.20-8.18 (dd, 1H, J=1.2; 7.8 Hz, C—H_{6′}), 8.10 (d, 1H, J=1.9 Hz, C—H4), 7.83-7.80 (dd, 1H, J=2.2; 8.9 Hz, C—H₆), 7.52-7.49 (d, 1H, J=8.9 Hz, C—H₇), 7.46 (dt, 1H, J=7.1 Hz, C—H_{4′}), 7.19 (s, 1H, C—H₁₀), 7.16-7.09 (m, 2H, C—H_{3′,5′}), 3.91 (s, 3H, OCH₃), 2.07 (s, 3H, NHCOCH₃). ¹³C NMR (75 Mhz, DMSO-d₆): δ 183.53 (C-3), 168.41 (C0), 161.21 (C-8), 158.32 (C-2′), 146.75 (C-2) 135.53 (C-5), 131.99 (C-4′), 131.11 (C-6′), 128.71 (C-6), 120.89 (C-1′), 120.68 (C-5′), 120.08 (C-9), 113.31 (C-3′), 113.15 (C-7), 111.57 (C-10), 105.47 (C-4), 55.84 (OCH₃), 23.83 (CH₃). Elemental analysis calcd (%) for C₁₈H₁₅NO₄: C, 69.89; H, 4.89; N, 4.53; found C, 69.87; H, 4.91; N, 4.52. m/z: 309.1001 (100.0%).

A2: (Z)-N-(2-(3-methoxybenzylidene)-3-oxo-2,3-dihydrobenzofuran-5-yl)acetamide

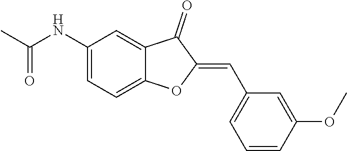 [SMILES: COc1cccc(/C=C2\Oc3ccc(NC(C)=O)cc3C2=O)c1]

Yield: 71%; ¹H NMR (300 MHz, DMSO-d₆): δ 10.16 (s, 1H, NH), 8.10 (d, 1H, J=2 Hz, C—H₄), 7.83-7.80 (dd, 1H, J=2.2; 8.9 Hz, C—H₆), 7.60-7.55 (m, 2H, C—H_{2′,4′}), 7.54-7.51 (d, 1H, J=8.9 Hz, C—H₇), 7.43 (dt, 1H, J=8.0 Hz, C—H_{5′}), 7.06-7.03 (dd, 1H, J=2.6; 8.2 Hz, C—H₆′), 6.90 (s, 1H, C—H₁₀), 7.16-7.09 (m, 2H, C—H_{3′,5′}), 3.82 (s, 3H, OCH₃), 2.07 (s, 3H, NHCOCH₃). ¹³C NMR (75 Mhz, DMSO-d₆): δ 183.68 (C-3), 168.42 (CO), 161.33 (C-8), 159.42 (C-3′), 146.88 (C-2), 135.58 (C-5), 133.08 (C-6′), 130 (C-2′), 128.82 (C-6), 123.74 (C-1′), 120.59 (C-9), 116.57 (C-6′), 115.76 (C-4′), 113.35 (C-7), 113.15 (C-4), 112.07 (C-10), 55.15 (OCH₃), 23.83 (CH₃). Elemental analysis calcd (%) for C₁₈H₁₅NO₄: C, 69.89; H, 4.89; N, 4.53; found C, 69.84; H, 4.88; N, 4.53. m/z: 309.1001 (100.0%).

A7: (Z)-N-(2-(3-(benzyloxy)benzylidene)-3-oxo-2,3-dihydrobenzofuran-5-yl)acetamide

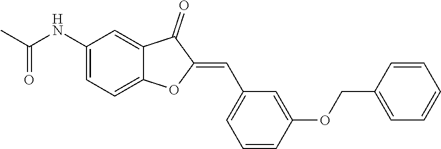 [SMILES: CC(=O)Nc1ccc2c(c1)C(=O)/C(=C/c1cccc(OCc3ccccc3)c1)O2]

Yield: 80%; ¹H NMR (300 MHz, DMSO-d₆): δ 10.16 (s, 1H, NH), 8.10 (d, 1H, J=2 Hz, C—H₄), 7.84-7.80 (dd, 1H, J=2.2; 8.9 Hz, C—H₆), 7.63 (bs, 1H, C—H_{2′}), 7.58-7.32 (m, 8H,), 7.14-7.11 (dd, 1H, J=7.9 Hz, C—H_{4′}), 6.89 (s, 1H, C—H₁₀), 5.18 (m, 2H, CH₂), 2.07 (s, 3H, NHCOCH₃). ¹³C NMR (75 Mhz, DMSO-d₆): δ 183.74 (C-4), 168.51 (CO), 161.36 (C-8), 158.53 (C-3′), 146.92 (C-2), 136.87 (C-1bn), 135.64 (C-5), 133.14 (C-5′), 130.1 (C-6′), 128.87 (C-6), 128.48 (C-3bn), 127.94 (C-4bn), 127.83 (C-2bn), 124.14 (C-1′), 120.62 (C-9), 117.27 (C-4′), 116.75 (C-2′), 113.45 (C-7), 113.17 (C-4), 112.09 (C-10), 69.34 (CH2), 23.91 (CH3). Elemental analysis calcd (%) for C₂₄H₁₉NO₄: C, 74.79; H, 4.97; N, 3.63; found C, 74.74; H, 5.01; N, 3.60. m/z: 385.13141 (100.0%).

A9: (Z)-5-amino-2-(3-(benzyloxy)benzylidene)benzofuran-3(2H)-one

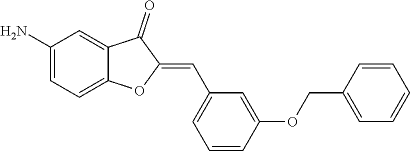 [SMILES: Nc1ccc2c(c1)C(=O)/C(=C/c1cccc(OCc3ccccc3)c1)O2]

Yield: 72%; ¹H NMR (300 MHz, DMSO-d₆): δ 7.61 (bs, 1H, C—H_{2′}), 7.55-7.53 (d, 1H, J=8.8 Hz, C—H₇), 7.50-7.48 (d, 2H, C—H_{2bn}), 7.41 (dt, 2H, C—H_{3bn}), 7.36-7.33 (m, 2H, C—H_{5′,4bn}), 7.11-7.09 (dd, 1H, J=7.9 Hz, C—H₆), 7.06-7.04 (dd, 1H, J=7.9 Hz, C—H_{4′}), 6.83 (d, 1H, J=2 Hz, C—H₄), 6.77 (s, 1H, C—H₁₀), 5.26 (bs, 2H, NH₂), 5.17 (m, 2H, CH₂). ¹³C NMR (75 Mhz, DMSO-d₆): δ 184.24 (C-3), 158.48 (C-3′), 158.14 (C-8), 147.21 (C-2), 145.63 (C-5), 136.87 (C-1bn), 133.43 (C-1′), 129.97 (C-5′), 128.42 (C-3bn), 127.86 (C-4bn), 127.75 (C-2bn), 124.71 (C-6), 123.89 (C-7), 120.91 (C-9), 117.04 (C-4′), 116.4 (C-2′), 113.21 (C-6′), 110.73 (C-10), 105.45 (C-4), 69.31 (CH2). Elemental analysis calcd (%) for C₂₂H₁₇NO₃: C, 76.95; H, 4.99; N, 4.08; found C, 76.88; H, 5.01; N, 4.04. m/z: 343.12084 (100.0%).

A12: (Z)-N-(3-oxo-2-(4-phenoxybenzylidene)-2,3-dihydrobenzofuran-5-yl)acetamide

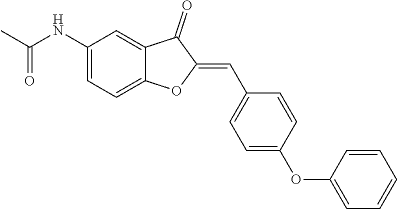 [SMILES: CC(=O)Nc1ccc2c(c1)C(=O)/C(=C/c1ccc(Oc3ccccc3)cc1)O2]

Yield: 67%; ¹H NMR (300 MHz, DMSO-d₆): δ 10.15 (s, 1H, NH), 8.11 (d, 1H, J=2 Hz, C—H₄), 8.03-8.00 (d, 2H, J=7.9 Hz, C—H_{2′}), 7.83-7.79 (dd, 1H, J=2.2; 8.9 Hz, C—H₆), 7.50-7.42 (m, 3H, C—H_{7,7′}), 7.22 (t, 1H, J=7.4 Hz, C—H_{8′}), 7.12-7.09 (m, 4H, C—H₃′,6′), 6.94 (s, 1H, C—H₁₀), 2.07 (s, 3H, NHCOCH₃). ¹³C NMR (75 Mhz, DMSO-d₆): δ 183.43 (C-3), 168.38 (CO), 161.14 (C-8), 158.41 (C-1″), 155.43 (C-4′), 146.18 (C-2), 135.5 (C-5), 133.43 (C-3″), 130.15 (C-2′), 128.67 (C-6), 126.79 (C-1′), 124.27 (C-4″), 120.76 (C-9), 119.41 (C-3′), 118.28 (C-2″), 113.24 (C-7), 113.11 (C-4), 111.83 (C-10), 23.82 (CH₃). Elemental analysis calcd (%) for C₂₃H₁₇NO₄: C, 74.38; H, 4.61; N, 3.77; found C, 74.35; H, 4.67; N, 3.73. m/z: 371.11576 (100.0%).

B1: (Z)-5-amino-2-(3-phenoxybenzylidene)benzofuran-3(2H)-one

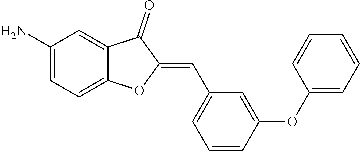 [SMILES: Nc1ccc2c(c1)C(=O)/C(=C/c1cccc(Oc3ccccc3)c1)O2]

Yield: 51%; ¹H NMR (300 MHz, DMSO-d₆): δ 7.71-7.69 (d, 1H, J=7.9 Hz, C—H_{6′}0 ), 7.64 (bs, 1H, C—H_{2′}), 7.51-7.49 (d, 1H, J=7.9 Hz, C—H₆), 7.44 (dt, 2H, C—H_{9′}), 7.22-7.15 (m, 2H, C—H_{4,5′}), 7.09-7.04 (m, 4H, C—H_{7,9′,10′}), 6.83 (d, 1H, J=2.02 Hz, C—H₄), 6.77 (s, 1H, C—H₁₀), 5.41 (bs, 2H, NH₂). ¹³C NMR (75 Mhz, DMSO-d₆): δ 184.24 (C-3), 158.13 (C-7′), 157.1 (C-3′), 156.21 (C-8), 147.41 (C-2), 145.56 (C-5), 134.09 (C-1′), 130.52 (C-5′), 130.15 (C-9′), 126.34 (C-6), 124.82 (C-9), 123.81 (C-6′), 120.88 (C-10′), 120.43 (C-4′), 119.65 (C-4), 118.95 (C-8′), 113.13 (C-8), 110.13 (C-10), 105.61 (C-7). Elemental analysis calcd (%) for C₂₁H₁₅NO₃: C, 76.58; H, 4.59; N, 4.25; found C, 76.56; H, 4.61; N, 4.22. m/z: 329.10519 (100.0%).

B4: (Z)-N-(2-(3-isopropoxybenzylidene)-3-oxo-2,3-dihydrobenzofuran-5-yl)acetamide

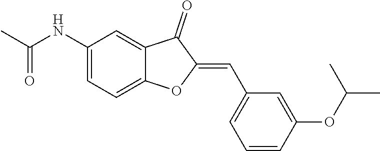 [SMILES: CC(=O)Nc1ccc2c(c1)C(=O)/C(=C/c1cccc(OC(C)C)c1)O2]

Yield: 91%; ¹H NMR (300 MHz, DMSO-d₆): δ 10.19 (s, 1H, NH), 8.12 (d, 1H, J=2 Hz, C—H₄), 7.83-7.80 (dd, 1H, J=2.2; 8.9 Hz, C—H₆), 7.57-7.52 (m, 3H, C—H_{2′,4′,7}), 7.40 (dt, 1H, J=8.0 Hz, C—H_{5′}), 7.04-7.01 (dd, 1H, J=2.6; 8.2 Hz, C—H₆′), 6.91 (s, 1H, C—H₁₀), 4.68 (q, 1H, J=5.9; 11.9 Hz, C—Hᵢₛₒₚ), 2.07 (s, 3H, NHCOCH₃), 1.31-1.29 (d, 6H, J=5.8 Hz, C—H3ᵢₛₒₚ). ¹³C NMR (75 Mhz, DMSO-d₆): δ 183.73 (C-3), 168.49 (CO), 161.35 (C-8), 157.66 (C-3′), 146.87 (C-2), 135.62 (C-5), 133.17 (C-1′), 130.1 (C-5′), 128.86 (C-6), 123.56 (C-6′), 120.64 (C-9), 118.25 (C-2′), 117.32 (C-3′), 113.41 (C-7), 113.17 (C-4), 112.28 (C-10), 69.35 (CHiPr), 23.88 (CH3), 21.77 (CH3iPr). Elemental analysis calcd (%) for C₂₀H₁₉NO₄: C, 71.20; H, 5.68; N, 4.15; found C, 71.18; H, 5.66; N, 4.16. m/z: m/z: 337.13 (100.0%).

B5: (Z)-N-(2-(4-isopropoxybenzylidene)-3-oxo-2,3-dihydrobenzofuran-5-yl)acetamide

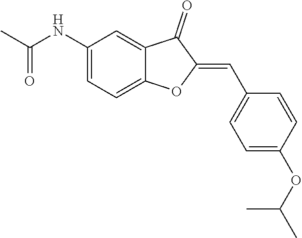 [SMILES: CC(=O)Nc1ccc2c(c1)C(=O)/C(=C/c1ccc(OC(C)C)cc1)O2]

Yield: 93%; mp: xx-xx ° C.; ¹H NMR (300 MHz, DMSO-d₆): δ 10.14 (s, 1H, NH), 8.10 (d, 1H, J=1.9 Hz, C—H₄), 7.94-7.91 (d, 2H, J=8.8 Hz, C—H_{2′}), 7.82-7.78 (dd, 1H, J=2.2; 8.8 Hz, C—H₆), 7.51-7.48 (d, 1H, J=8.89 Hz, C—H₇), 7.06-7.03 (d, 2H, J=8.8 Hz, C—H_{3′}), 6.90 (s, 1H, C—H₁₀), 4.72 (q, 1H, J=5.9; 11.9 Hz, C—Hᵢₛₒₚ), 2.07 (s, 3H, NHCOCH₃), 1.30-1.28 (d, 6H, J=5.8 Hz, C—H3ᵢₛₒₚ). ¹³C NMR (75 Mhz, DMSO-d₆): δ 183.25 (C-3), 168.39 (CO), 160.98 (C-8), 159.16 (C-4′), 145.6 (C-2), 135.39 (C-5), 133.41 (C-2′), 128.48 (C-6), 124.02 (C-1′), 120.94 (C-9), 115.94 (C-3′), 113.2 (C-7), 113.06 (C-4), 112.77 (C-10), 69.5 (CHiPr), 23.83 (CH3), 21.68 (CH3iPr). Elemental analysis calcd (%) for C₂₀H₁₉NO₄: C, 71.20; H, 5.68; N, 4.15; found C, 71.18; H, 5.69; N, 4.12. m/z: m/z: 337.13 (100.0%).

B10: (Z)-N-(2-(3-fluorobenzylidene)-3-oxo-2,3-dihydrobenzofuran-5-yl)acetamide

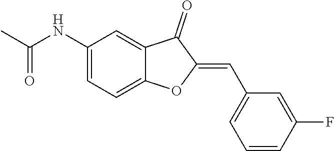 [SMILES: CC(=O)Nc1ccc2c(c1)C(=O)/C(=C/c1cccc(F)c1)O2]

Yield: 82%; ¹H NMR (300 MHz, DMSO-d₆): δ 10.22 (s, 1h, NH), 8.12 (d, 1H, J=2.1 Hz, C—H₄), 7.83-7.79 (m, 3H, C—H_{6,2′,6′}), 7.59-7.51 (m, 2H, C—H_{4′,7}), 7.31-7.39 (dt, 1H, J=2.1, 8.4 Hz, C—H_{5′}), 6.59 (s, 1H, C—H₁₀), 2.07 (s, 3H, CH₃). ¹³C NMR (75 Mhz, DMSO-d₆): δ 183.83 (C-3), 168.6 (CO), 163.85-160.62 (C-3′, J=244 Hz), 161.48 (C-8), 147.37 (C-2), 135.77 (C-5), 134.3-134.19 (C-1′, J=8.25 Hz), 131.08-130.97 (C-5′, J=8.25 Hz), 129.05 (C-6), 127.62-127.58 (C-6′, J=2.75 Hz), 120.53 (C-9), 117.45-117.15 (C-4′, J=22.56 Hz), 117.02-116.73 (C-2′, J=21.5 Hz), 113.53 (C-7), 113.25 (C-4), 110.69-110.66 (C-10, J=2.75 Hz), 23.92 (CH3). Elemental analysis calcd (%) for C₁₇H₁₂FNO₃: C, 68.68; H, 4.07; N, 4.71; found C, 68.58; H, 4.12; N, 4.73. m/z: 297.08 (100.0%).

B11: (Z)-5-amino-2-(2-fluorobenzylidene)benzofuran-3(2H)-one

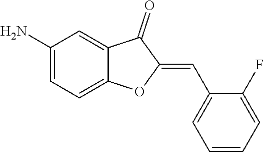 [SMILES: Nc1ccc2c(c1)C(=O)/C(=C/c1ccccc1F)O2]

Yield: 62%; ¹H NMR (300 MHz, DMSO-d₆): δ 8.23 (dt, 1H, J=1.65, 7.8 Hz, C—H_{6′}), 7.51-7.47 (m, 1H, C—H_{4′}), 7.37 (t, 1H, C—H_{3′}), 7.34 (dt, 1H, C—H_{5′}), 7.28-7.25 (d, 1H, J=8.8 Hz, C—H₇), 7.09-7.05 (dd, 1H, J=2.5, 8.7 Hz, C—H₆), 6.86 (d, 1H, J=2.4 Hz, C—H₄), 6.81 (s, 1H, C—H₁₀), 5.28 (bs, 2H, NH₂). ¹³C NMR (75 Mhz, DMSO-d₆): δ 184.05 (C-3), 164.75-161.71 (C-2′, J=260 Hz), 158.19 (C-8), 148.04 (C-2), 145.83 (C-5), 131.84-131.73 (C-4′, J=8 Hz), 131.2 (C-6′), 125.11-125.06 (C-5′, J=3 Hz), 124.84 (C-6), 120.69 (C-9), 119.97-119.81 (C-1′, J=12 Hz), 115.85-115.56 (C-3′), 113.22 (C-7), 105.54 (C-4), 100.82-100.72 (C-10). Elemental analysis calcd (%) for C₁₅H₁₀FNO₂: C, 70.58; H, 3.95; N, 5.49; found C, 70.44; H, 3.99; N, 5.32. m/z: 255.07 (100.0%).

C4: (Z)-4-((5-acetamido-3-oxobenzofuran-2(3H)-ylidene)methyl)benzoic acid

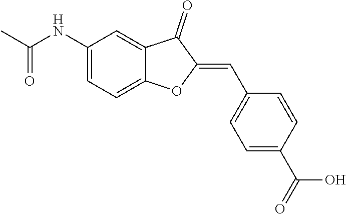 [SMILES: CC(=O)Nc1ccc2c(c1)C(=O)/C(=C/c1ccc(C(=O)O)cc1)O2]

Yield: 71%; ¹H NMR (300 MHz, DMSO-d₆): δ 10.28 (s, 1H, NH), 8.14 (d, 1H, J=1.9 Hz, C—H₄), 8.10-8.07 (d, 2H, J=8.8 Hz, C—H_{2′}), 8.05-8.02 (d, 2H, J=8.8 Hz, C—H_{3′}), 7.87-7.83 (dd, 1H, J=2.2; 8.8 Hz, C—H₆), 7.55-7.53 (d, 1H, J=8.89 Hz, C—H₇), 6.98 (s, 1H, C—H₁₀), 2.08 (s, 3H, NHCOCH₃). ¹³C NMR (75 Mhz, DMSO-d₆): δ 183.85 (C-3), 168.54 (CO), 166.77 (COOH), 161.47 (C-8), 147.69 (C-2), 136.08 (C-1′), 135.82 (C-5), 131.35 (C-4′), 131.23 (C-3′), 129.74 (C-2′), 129.04 (C-6), 120.47 (C-9), 113.44 (C-7), 113.25 (C-4), 110.61 (C-10), 23.89 (CH3). Elemental analysis calcd (%) for C₁₈H₁₃NO₅: C, 66.87; H, 4.05; N, 4.33; found C, 66.85; H, 4.12; N, 4.27. m/z: 323.07937 (100.0%).

C8: (Z)-N-(7-nitro-3-oxo-2-(3-phenoxybenzylidene)-2,3-dihydrobenzofuran-5-yl) acetamide

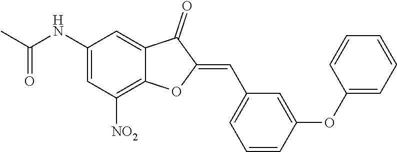 [SMILES: CC(=O)Nc1cc2c(c([N+](=O)[O-])c1)O/C(=C\c1cccc(Oc3ccccc3)c1)C2=O]

Yield: 91%; ¹H NMR (300 MHz, DMSO-d₆): δ 10.51 (bs, 1H, NH), 8.71 (d, 1H, J=2.2 Hz, C—H₆), 8.35 (d, 1H, J=2.2 Hz, C—H₄), 7.89-7.86 (d, 2H, J=7.8 Hz, C—H_{2″}), 7.56 (t, 1H, J=8.16 Hz, C—H_{5′}), 7.42 (dt, 2H, C—H₃″), 7.20-7.17 (d, 1H, J=7.3 Hz, C—H_{4′}), 7.14 (s, 1H, C—H_{2′}), 7.09-7.06 (m, 2H, C—H_{10, 6′}), 2.11 (s, 3H, CH₃). ¹³C NMR (75 Mhz, DMSO-d₆): δ 178.66 (C-3), 169.01 (CO), 157.07 (C-1″), 156.36 (C-3′), 153.21 (C-8), 146.13 (C-2), 143.98 (C-7), 135.31 (C-5), 133.21 (C-1′), 130.64 (C-5′), 130.09 (C-3″), 127.01 (C-6), 124.60 (C-9), 123.69 (C-4″), 121.59 (C-6′), 120.86 (C-4), 119.76 (C-4′), 119.35 (C-2′), 118.66 (C-2″), 113.73 (C-10), 23.86 (CH3). Elemental analysis calcd (%) for C₂₃H₁₆N₂O₆: C, 66.34; H, 3.87; N, 6.73; found C, 66.21; H, 3.74; N, 6.71. m/z: 416.10084 (100.0%).

C10: (Z)-2-benzylidene-6-hydroxy-4-methoxy-5,7-dimethylbenzofuran-3(2H)-one

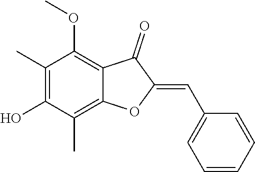 [SMILES: COc1c(C)c(O)c(C)c2c1C(=O)/C(=C/c1ccccc1)O2]

Yield: 68%; ¹H NMR (300 MHz, DMSO-d₆): δ 7.95-7.93 (d, 2H, J=7.4 Hz, C—H_{2′}), 7.50 (t, 2H, J=7.3 Hz, C—H_{3′}), 7.42 (t, 1H, J=7.3 Hz, C—H_{4′}), 6.71 (s, 1H, C—H₁₀), 4.01 (s, 3H, OCH₃), 2.23 (s, 3H, CH₃), 2.04 (s, 3H, CH₃). ¹³C NMR (75 Mhz, DMSO-d₆): δ 179.45 (C-3), 163.77 (C-6), 163.16 (C-8), 154.54 (C-4), 147.42 (C-2), 132.36 (C-1′), 130.74 (C-2′), 129.26 (C-4′), 128.94 (C-3′), 111.82 (C-5), 109.05 (C-10), 105.08 (C-9), 102.86 (C-7), 61.24 (OCH₃), 8.71 (CH₃), 8.10 (CH₃). Elemental analysis calcd (%) for C₁₈H₁₆O₄: C, 72.96; H, 5.44; found C, 72.78; H, 5.51. m/z: 296.10486 (100.0%).

C11: (Z)-6-amino-2-(4-methoxybenzylidene)benzofuran-3(2H)-one

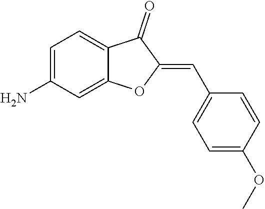 [SMILES: COc1ccc(/C=C2\Oc3cc(N)ccc3C2=O)cc1]

Yield: 52%; ¹H NMR (300 MHz, DMSO-d₆): δ 7.87-7.84 (d, 2H, J=7.9 Hz, C—H_{2′}), 7.41-7.38 (d, 1H, J=8.25 Hz, C—H₄), 7.06-7.03 (d, 2H, J=7.4 Hz, C—H_{3′}), 6.59 (bs, 1H, C—H₇), 6.47-6.44 (dd, 1H, J=8.5 Hz, C—H₅), 6.41 (s, 1H, C—H₁₀), 3.82 (s, 3H, OCH₃). ¹³C NMR (75 Mhz, DMSO-d₆): δ 179.72 (C-4), 168.04 (C-8), 159.96 (C-6), 158.02 (C-4′), 146.99 (C-2), 132.36 (C-2′), 125.59 (C-4), 124.99 (C-1′), 114.47 (C-3′), 110.99 (C-10), 109.13 (C-9), 108.49 (C-5), 93.52 (C-7), 55.24 (OCH3). Elemental analysis calcd (%) for C₁₆H₁₃NO₃: C, 71.90; H, 4.90; N, 5.24; found C, 71.78; H, 4.97; N, 5.21. m/z: 267.08954 (100.0%).

C12: (Z)-2-(2,4-bis(benzyloxy)benzylidene)benzofuran-3(2H)-one

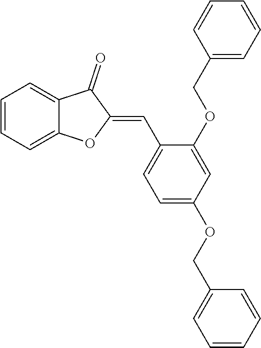 [SMILES: O=C1/C(=C/c2ccc(OCc3ccccc3)cc2OCc2ccccc2)Oc2ccccc21]

Yield: 79¹H NMR (300 MHz, DMSO-d₆): δ 8.22-8.20 (d, 1H, J=8.0 Hz, C—H_{6′}), 7.78-7.75 (m, 2H, C—H_{4,6}), 7.55-7.53 (d, 1H, J=8.5 Hz, C—H₇), 7.48-7.30 (m, 11H), 7.20 (s, 1H, C—H₁₀), 6.91 (d, 1H, J=2 Hz, C—H_{3′}), 6.84-6.81 (d, 1H, J=7.8 Hz, C—H_{5′}), 5.26 (s, 2H, CH₂), 5.20 (s, 2H, CH2). ¹³C NMR (75 Mhz, DMSO-d₆): δ 182.97 (C-3), 164.92 (C-8), 161.66 (C-4′), 159.09 (C-2′), 145.17 (C-2), 137.08 (C-1′), 136.44 (C-1Bn), 136.42 (C-1Bn), 132.6 (C-6), 128.55 (C-3Bn), 128.43 (C-3Bn), 128.05 (C-4bn), 127.98 (C-4Bn), 127.83 (C-2Bn), 127.72 (C-2Bn), 124.04 (C-4), 123.69 (C-5), 121.16 (C-9), 113.54 (C-7), 113.1 (C-5′), 107.81 (C-6′), 106.07 (C-10), 100.46 (C-3′), 70.06 (CH2), 69.64 (CH2). Elemental analysis calcd (%) for C₂₉H₂₂O₄: C, 80.17; H, 5.10; found C, 80.22; H, 5.11. m/z: 434.15181 (100.0%).

D1: (Z)-N-(3-oxo-2-(3,4,5-trimethoxybenzylidene)-2,3-dihydrobenzofuran-5-yl)acetamide [1]

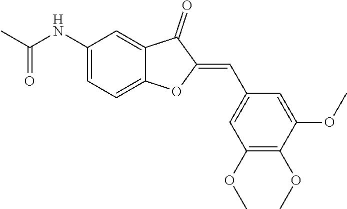 [SMILES: COc1cc(/C=C2\Oc3ccc(NC(C)=O)cc3C2=O)cc(OC)c1OC]

Yield: 93%; mp: 254° C. [1]; ¹H NMR (300 MHz, DMSO-d₆): δ 10.15 (s, 1H, NH), 8.09 (d, 1H, J=2.2 Hz, C—H₄), 7.84-7.81 (dd, 1H, J=2.2; 8.8 Hz, C—H₆), 7.54-7.51 (d, 1H, J=8.9 Hz, C—H₇), 7.36 (s, 2H, C—H_{2′}), 6.89 (s, 1H, C—H₁₀), 3.86 (s, 6H, OCH₃), 3.75 (s, 3H, OCH₃), 2.01 (s, 3H, CH₃). ¹³C NMR (75 Mhz, DMSO-d₆): δ 183.45 (C-3), 168.41 (CO), 161.14 (C-8), 152.96 (C-3′), 146.26 (C-2), 139.59 (C-4′), 135.53 (C-5), 128.63 (C-6), 127.25 (C-1′), 120.7 (C-9), 113.41 (C-7), 113.11 (C-4), 112.71 (C-10), 109.21 (C-2′), 60.16 (OCH3), 55.99 (OCH3), 23.84 (CH3). Elemental analysis calcd (%) for C₂₀H₁₉NO₆: C, 65.03; H, 5.18; N, 3.79; found C, 64.97; H, 5.21; N, 3.78. m/z: 369,12 (100.0%).

D2: (Z)-5-amino-2-(3,4,5-trimethoxybenzylidene)benzofuran-3(2H)-one

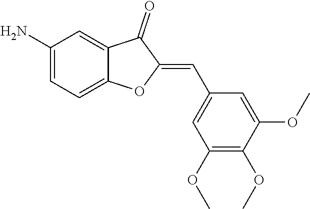 [SMILES: COc1cc(/C=C2\Oc3ccc(N)cc3C2=O)cc(OC)c1OC]

Yield: 56%; ¹H NMR (300 MHz, DMSO-d₆): δ 7.33 (s, 2H, C—H2′), 7.28-7.25 (d, 1H, J=8.9 Hz, C—H₇), 7.06-7.03 (dd, 1H, J=2.2; 8.8 Hz, C—H₆), 6.83 (d, 1H, J=2.2 Hz, C—H₄), 6.78 (s, 1H, C—H₁₀), 5.23 (bs, 2H, NH₂), 3.85 (s, 6H, OCH₃), 3.74 (s, 3H, OCH₃). ¹³C NMR (75 Mhz, DMSO-d₆): δ 184.01 (C-3), 157.94 (C-8), 152.93 (C-3′), 146.56 (C-2), 145.55 (C-5), 139.26 (C-4′), 127.59 (C-1′), 124.5 (C-6), 121.01 (C-9), 113.24 (C-7), 111.37 (C-10), 108.96 (C-2′), 105.36 (C-4), 60.13 (OCH3), 55.96 (OCH3). Elemental analysis calcd (%) for C₁₈H₁₇NO₅: C, 66.05; H, 5.23; N, 4.28; found C, 65.95; H, 5.28; N, 4.21. m/z: 327,11 (100.0%).

D3: (Z)-N-(3-oxo-2-(2,4,5-trimethoxybenzylidene)-2,3-dihydrobenzofuran-5-yl)acetamide

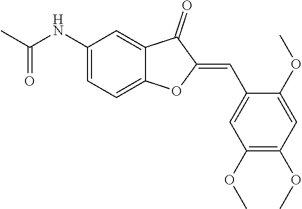 [SMILES: COc1cc(OC)c(OC)cc1/C=C1\Oc2ccc(NC(C)=O)cc2C1=O]

Yield: 54%; ¹H NMR (300 MHz, DMSO-d₆): δ 10.14 (s, 1H, NH), 8.06 (d, 1H, J=2.2 Hz, C—H₄), 7.82-7.77 (m, 2H, C—H_{6,3′}), 7.52-7.49 (d, 1H, J=8.9 Hz, C—H₇), 7.16 (s, 1H, C—H_{6′}), 6.78 (s, 1H, C—H₁₀), 3.92 (s, 3H, OCH₃), 3.89 (s, 3H, OCH₃), 3.82 (s, 3H, OCH₃), 2.07 (s, 3H, CH₃). ¹³C NMR (75 Mhz, DMSO-d₆): δ 185.87 (C-3), 168.38 (CO), 160.65 (C-8), 154.92 (C-2′), 152.78 (C-4′), 145.29 (C-2), 142.89 (C-5′), 135.34 (C-5), 128.17 (C-6), 121 (C-9), 114.09 (C-1′), 113.29 (C-7), 112.95 (C-4), 111.41 (C-10), 106.44 (C-6′), 97.39 (C-3′), 56.5 (OCH3), 56.21 (OCH3), 55.83 (OCH3), 23.83 (CH3). Elemental analysis calcd (%) for C₂₀H₁₉NO₆: C, 65.03; H, 5.18; N, 3.79; found C, 65.11; H, 5.22; N, 3.89. m/z: 369.12 (100.0%).

D8: (Z)-N-(2-benzylidene-3-oxo-2,3-dihydrobenzofuran-5-yl)acetamide [1]

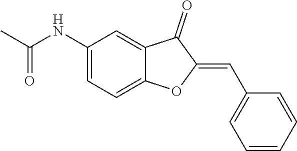 [SMILES: CC(=O)Nc1ccc2c(c1)C(=O)/C(=C/c1ccccc1)O2]

Yield: 63%; mp: 241° C. [1]; ¹H NMR (300 MHz, DMSO-d₆): δ 10.16 (s, 1H, NH), 8.17 (d, 1H, J=2 Hz, C—H_{4′}), 7.95-7.97 (d, 2H, J=6.8 Hz, C—H_{2′}), 7.83-7.77 (dd, 1H, J=2.2; 8.8 Hz, C—H₆), 7.53-7.48 (m, 4H, C—H_{7,3′,4′}), 2.07 (s, 3H, CH3). ¹³C NMR (75 MHz, DMSO-d₆): δ 182.09 (C-3), 168.89 (COCH3), 164.67 (C-8), 146.90 (C-2), 139.50 (C-5), 132.30 (C-4′), 130.76 (C-2′), 128.72 (C-3′), 128.53 (C-6), 128.07 (C-1′), 120.18 (C-9), 114.21 (C-7), 111.60 (C-4), 109.20 (C-10), 24.08 (CH3). Elemental analysis calcd (%) for C₁₇H₁₃NO₃: C, 73.11; H, 4.69; N, 5.02; found C, 73.08; H, 4.75; N, 4.95. m/z: 279.09 (100.0%).

D10: (Z)-2-(4-methoxybenzylidene)benzofuran-3(2H)-one [2]

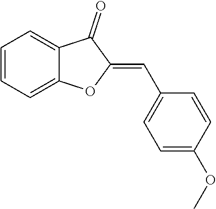 [SMILES: COc1ccc(/C=C2\Oc3ccccc3C2=O)cc1]

Yield: 92%; mp: 138° C.; 1H NMR (300 MHz, CDCl₃) δ 7.88 (d, J =8.7 Hz, 2H), 7.79 (d, J=7.5 Hz, 1H), 7.65-7.60 (m, 1H), 7.31 (d, J=8.3 Hz, 1H), 7.20 (t, J=7.4 Hz, 1H), 6.97 (d, J=8.8 Hz, 2H), 6.87 (s, 1H), 3.86 (s, 3H). ¹³C NMR (75 MHz, CDCl₃) δ 184.54 (C-3), 165.84 (C-8), 161.09 (C-4′), 145.89 (C-2), 136.52 (C-6), 133.45 (C-2′), 125.07 (C-1′), 124.55 (C-4), 123.27 (C-5), 121.96 (C-9), 114.51 (C-7), 113.41 (C-3′), 112.88 (C-10), 55.39 (OCH3). Elemental analysis calcd (%) for C₁₆H₁₂O₃: C, 76.18; H, 4.79; found C, 76.11; H, 4.83. m/z: 252.07864 (100.0%).

E5: (Z)-6-(4-((3-oxobenzofuran-2(3H)-ylidene)methyl)phenoxy)-2-phenyl-4H-chromen-4-one

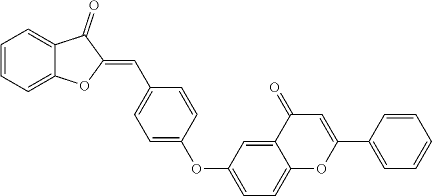 [SMILES: O=C1/C(=C/c2ccc(Oc3ccc4oc(-c5ccccc5)cc(=O)c4c3)cc2)Oc2ccccc21]

MR885

Yield: 58%; ¹H NMR (300 MHz, CD-Cl₃) δ 8.26-8.23 (d, 1H, C—H_{4″}), 7.96-7.94 (d, 4H, C—H_{2′″,2′}), 7.87 81 (d, 1H, J=2.2 Hz, C—H₅), 7.72 (dt, 1H, C—H_{6″}), 7.68-7.66 (d, 1H, C—H₈), 7.58-7.55 (m, H), 7.51-7.48 (dd, 1H, J=2.2; 8.5 Hz, C—H₇), 7.44 (t, 1H, C—H_{5″}), 7.18-7.16 (d, 2H, C—H_{3″}), 6.85 (s, 1H, C—H₃), 6.80 (s, 1H, C—Hₐᵤ). ¹³C NMR (75 Mhz, CD-Cl₃) δ 177.65 (C-4), 177.07 (C-3″), 163.10 (C-8″), 162.28 (C-2), 159.39 (C-4″'), 155.60 (C-9), 152.69 (C-6), 152.18 (C-2″), 133.36 (C-6″), 131.32 (C-4′), 130.89 (C-1′), 128.61 (C-2″'), 128.56 (C-3′), 127.88 (C-2′), 126.28 (C-1″'), 125.78 (C-3″'), 125.73 (C-4″), 124.90 (C-7), 124.74 (C-5″), 124.47 (C-9″), 123.25 (C-10), 119.94 (C-8), 118.25 (C-7″), 117.63 (C-5), 113.62 (CH), 106.30 (C-3). Elemental analysis calcd (%) for C₃₀H₁₈O₅: C, 78.59; H, 3.96; found C, 78.75; H, 4.05; m/z: 458.11542 (100.0%).

F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(242,4(8),9,13,15(20),16,18-octaen-13-ylium bromide

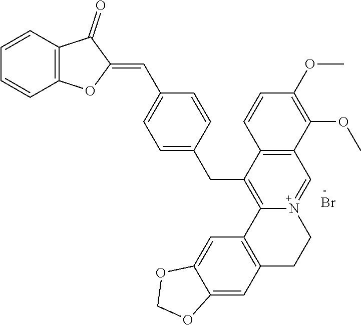

Yield 90%. ¹H RMN (300 MHz, DMSO-d6) δ ppm: 10.06 (1H, s, H-8), 8.13 (1H, brd, J=7.8, H-7″), 8.11 (1H, d, J=9.4 Hz, H-11), 8.09 (2H, d, J=8.1 Hz, H-4′), 8.04 (1H, brd, J=7.8 Hz, H-4″), 7.81 (1H, d, J=9.4 Hz, H-12), 7.55 (1H, brt, J=7.8 Hz, H-5″), 7.47(1H, brt, J=7.8Hz, H-6″), 7.39 (2H, d, J=8.1Hz, H3′), 7.18 (1H, s, H-4), 6.99 (1H, s, H-1), 6.08 (2H, s, —OCH2O—), 4.90 (2H, brs, H-6), 4.86 (2H, s, H-1′), 4.14 (3H, s, OCH3-9), 4.03 (3H, s, OCH3-10), 3.19 (3H, t, J=5.3 Hz, H-5). ¹³C RMN (300 MHz, DMSO-d6) δ ppm: 183.55 (C-8′), 165.38 (C-14′), 150.19 (C-10′), 149.20 (C-3), 146.40 (C-2), 146.34 (C-7′), 145.51 (C-8), 144.27 (C-9), 141.45 (C-2′), 137.73 (C-12′), 137.17 (C-13a), 134.05 (C-4a), 132.64 (C-12a), 131.98 (C-3′), 130.48 (C-5′), 129.62 (C-13), 128.78 (C-4′), 126.15 (C-12), 124.29 (C-10′), 124.01 (C-11′), 121.59 (C-11), 121.27 (C-8a), 120.81 (C-9′), 119.93 (C-13b), 111.68 (C-6′), 108.48 (C-4), 108.19 (C-1), 102.06 (—OCH2O—),62.04 (OCH3-C-9), 56.90 (OCH3-C-10), 56.90 (C-6), 35.55 (C-1′), 27.23 (C-5). Elemental analysis calcd (%) for C₃₆H₂₈BrNO₆: C, 66.47; H, 4.34; N, 2.15; found C, 66.25; H, 4.86; N, 2.11. m/z: 649.11000 (100.0%).

G4: (Z)-N-(2-(4-methoxybenzylidene)-3-oxo-2,3-dihydrobenzofuran-6-yl)acetamide

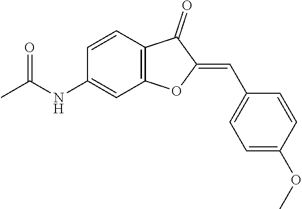 [SMILES: COc1ccc(/C=C2\Oc3cc(NC(C)=O)ccc3C2=O)cc1]

Yield: 67%; ¹H NMR (300 MHz, DMSO-d₆): δ 10.59 (s, 1H, NH), 8.00 (d, 1H, J=2.0 Hz, C—H₇), 7.94-7.91 (d, 2H, J=8.4 Hz, C—H_{2′}), 7.70-7.67 (d, 1H, J=8.0 Hz, C—H₄), 7.24-7.21 (dd, 1H, J=2.0; 8.0 Hz, C—H₅), 7.08-7.05 (d, 2H, J=8.4 Hz, C—H_{3′}), 6.81 (s, 1H, C—H₁₀), 3.82 (s, 3H, OCH3), 2.13 (s, 3H, CH3). ¹³C NMR (75 MHz, DMSO-d₆): δ 181.72 (C-3), 169.69 (CO), 166.57 (C-8), 160.72 (C-4′), 147.31 (C-6), 146.02 (C-2), 133.29 (C-2′), 125.03 (C-4), 124.56 (C-1′), 115.8 (C-9), 114.76 (C-3′, C-5), 111.72 (C-10), 101.51 (C-7), 55.43 (OCH3), 24.4 (CH3). Elemental analysis calcd (%) for C₁₈H₁₅NO₄: C, 69.89; H, 4.89; N, 4.53; found C, 69.89; H, 4.89; N, 4.53. m/z: 309.31600 (100.0%).

MR1042: (Z)-2-(4-(bromomethyl)benzylidene)benzofuran-3(2H)-one

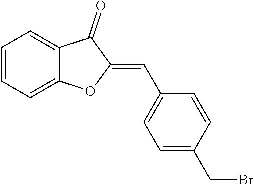 [SMILES: O=C1/C(=C/c2ccc(CBr)cc2)Oc2ccccc21]

Yield: 77%; ¹H NMR (300 MHz, DMSO-d₆): δ 7.90-7.87 (d, 2H, J=8.4 Hz, C—H_{2′}), 7.81-7.77 (m, 2H, C—H_{4,6}), 7.54 (d, 1H, J=8.5 Hz, C—H₅), 7.31 (t, 1H, J=7.6 Hz, C—H₇), 7.18-7.15 (2H, d, J=8.4 Hz, C—H_{3′}), 6.90 (s, 1H, C—H₁₀), 4.52 (s, 2H, CH2). ¹³C NMR (CDCl₃, 75 MHz) δ 182.21 (C-3), 164.52 (C-8), 144.69 (C-2), 137.25 (C-4′), 137.02 (C-6), 132.12 (C-2′), 124.12 (C-4), 123.35 (C-5), 122.89 (C-1′), 121.24 (C-9), 117.18 (C-3′), 113.48 (C-7), 112.12 (C-10), 33.24 (CH2). Elemental analysis calcd (%) for C₁₆H₁₁BrO₂: C, 60.98; H, 3.52; found C, 60.84; H, 3.58. m/z: 313.99424 (100.0%).

AD-1-44: (Z)-N-(2-(3,4-bis(benzyloxy)benzylidene)-3-oxo-2,3-dihydrobenzofuran-5-yl)acetamide

Starting from chalcone with Hg(Ac)₂/pyridine at 110° C. for 1 h.

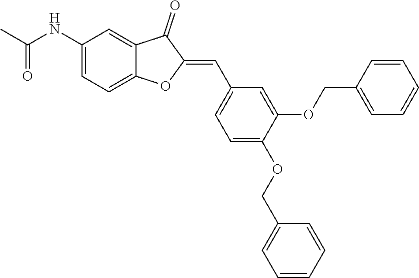 [SMILES: CC(=O)Nc1ccc2c(c1)C(=O)/C(=C/c1ccc(OCc3ccccc3)c(OCc3ccccc3)c1)O2]

Yield: 54%; 1H NMR (300 MHz, DMSO d6): δ 10.17 (s, 1H, NH), 8.09 (d, 1H, J=2.1 Hz, C—H4), 7.84-7.80 (dd, 1H, J=1.8;8.6 Hz, C—H6), 7.72 (d, 1H, J=1.8 Hz, C—H2′), 7;58-7.55 (dd, 1H, J=1.9; 8.6 Hz, C—H6′), 7.54-7.32 (m, 11H, C—H benzyl, C—H7), 7.22 (d, 1H, J=8.5 Hz, C—H5′), 6.86 (s, 1H, C—H10), 5.24 (bs, 4H, CH2 bn), 2.07 (s, 3H, CH3). 13C NMR (75 MHz, DMSO d6): δ 183.18 (C-3), 168.55 (NHCO), 161.76 (C-8), 160.93 (C-4′), 159.15 (C-3′), 145.78 (C-2), 136.51 (C-1bn), 136.48 (C-1bn) 135.45 (C-5), 132.7 (C-6), 128.67 (C-3bn), 128.55 (C-3bn), 128.48 (C-7), 128.18 (C-4bn), 128.11 (C-4bn), 127.97 (C-2bn), 127.83 (C-2bn), 121.05 (C-9), 113.62 (C-4), 113.34 (C-5′), 107.89 (C-6′), 106.21 (C-10), 100.49 (C-2′), 70.12 (CH2), 69.72 (CH2), 23.91 (CH3).

Elemental analysis calcd (%) for C₃₁H₂₅NO₅: C, 75.75; H, 5.13; N, 2.85; found C, 75.71; H, 5.09; N, 2.86. m/z: 491.17 (100.0%).

MR1065: (Z)-2-(3,4-bis(benzyloxy)benzylidene)-3-oxo-2,3-dihydrobenzofuran-5-carboxylic acid

Starting from chalcone with Hg(Ac)₂/pyridine at 110° C. for 1 h.

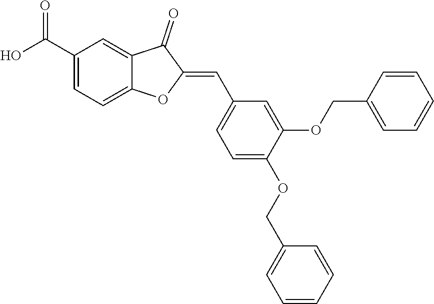 [SMILES: O=C(O)c1ccc2c(c1)C(=O)/C(=C/c1ccc(OCc3ccccc3)c(OCc3ccccc3)c1)O2]

Yield: 64%; 1H NMR (300 MHz, DMSO d6): δ 8.35-8.34-8.30 (dd, 1H, J=1.8; 8.6 Hz, C—H6), 8.22 (d, 1H, J=1.4 Hz, C—H4), 7.72 (d, 1H, J=1.8 Hz, C—H2′), 7.66-7.63 (d, 1H, J=8.6 Hz, C—H7), 7.58-7.53 (dd, 1H, J=1.8; 8.6 Hz, C—H6′), 7.50-7.30 (m, 10H, C—H benzyl), 7.20-7.17 (d, 1H, J=8.6 Hz, C—H5′), 6.93 (s, 1H, C—H10), 5.23 (bs, 4H, CH2).

13C NMR (75 MHz, DMSO d6): δ 182.45 (C-3), 167.24 (COOH), 166.2 (C-8), 150.53 (C-3′), 148.08 (C-4′), 145.49 (C-2), 137.98(C-6), 137.06 (C-1bn), 136.74 (C-1bn), 128.49 (C-3bn), 127.97 (C-4bn), 127.9 (C-4bn), 127.66 (C-2bn), 127.58 (C-2bn), 126.54 (C-1′), 126.46 (C-4), 125.38 (C-6′), 124.52 (C-9), 121.37 (C-5), 116.77 (C-5′), 114.08 (C-7), 114.07 (C-2′), 113.58 (C-10), 70.08 (CH2), 69.92 (CH2).

Elemental analysis calcd (%) for C₃₀H₂₂O₆: C, 75.30; H, 4.63; found C, 75.23; H, 4.68. m/z: 478.14 (100.0%).

AD-1-61: (2Z)-2-(3-methoxybenzylidene)-3-oxo-2,3-dihydro-1-benzofuran-5-carboxylic acid

Starting from chalcone with Hg(Ac)₂/pyridine at 110° C. for 1 h.

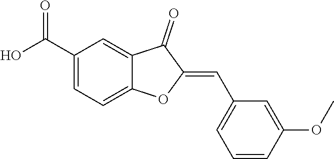 [SMILES: COc1cccc(/C=C2\Oc3ccc(C(=O)O)cc3C2=O)c1]

Yield: 83%; 1H NMR (300 MHz, DMSO d6): δ 13.27 (bs, 1H, COOH), 8.35-8.32 (dd, 1H, J=1.8; 8.6 Hz, C—H6), 8.25 (d, 1H, J=1.4 Hz, C—H4), 7.71-7.68 (d, 1H, J=8.2 Hz, C—H7), 7.64-7.61 (d, 1H, J=7.8 Hz, C—H6′), 7.58 (d, 1H, J=2.3 Hz, C—H2′), 7.45 (t, 1H, J=8.1 Hz, C—H5′), 7.09-7.06 (dd, 1H, J=1.8, 8.2 Hz, C—H4′), 7.01 (s, 1H, C-H10), 3.83 (s, 3H, OCH3). 13C NMR (75 MHz, DMSO d6): δ 182.94 (C-3), 167.63 (COOH), 166.08 (C-8), 159.47 (C-3′), 146.64 (C-2), 138.30 (C-6), 132.81 (C-1′), 130.12 (C-5′), 126.77 (C-5), 125.45 (C-4), 123.96 (C-6′), 121.12 (C-9), 116.71 (C-4′), 116.19 (C-2′), 113.64 (C-7), 113.21 (C-10), 55.21 (CH3). Elemental analysis calcd (%) for C₁₇H₁₂O₅: C, 68.92; H, 4.08; found C, 68.87; H, 4.11. m/z: 296.07 (100.0%).

AD-1-62: (2Z)-2-[4-(dimethylamino)benzylidene]-3-oxo-2,3-dihydro-1-benzofuran-5-carboxylic acid

Starting from chalcone with Hg(Ac)₂/pyridine at 110° C. for 1 h.

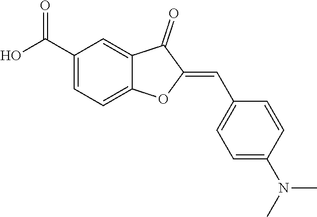 [SMILES: CN(C)c1ccc(/C=C2\Oc3ccc(C(=O)O)cc3C2=O)cc1]

Yield: 72%; 1H NMR (300 MHz, DMSO d6): δ 8.28-8.25 (dd, 1H, J=1.8; 8.6 Hz, C—H6), 8.20 (d, 1H, J=1.4 Hz, C—H4), 7.86-7.83 (d, 2H, J=8.9 Hz, C-H2′), 7.61-7.58 (d, 1H, J=8.2 Hz, C—H7), 6.93 (s, 1H, C—H10), 6.81-6.78 (d, 2H, J=8.9 Hz, C—H3′). 13C NMR (75 MHz, DMSO d6): δ 181.61 (C-3), 166.65 (COOH), 166.54 (C-8), 151.71 (C-4′), 144.38 (C-2), 137.28 (C-6), 133.89 (C-2′), 126.77 (C-1′), 125.18 (C-5), 121.99 (C-9), 118.59 (C-7), 116.05 (C-4), 113.36 (C-10), 112.1 (C-3′), 39.66 (CH3). Elemental analysis calcd (%) for C₁₈H₁₅NO₄: C, 69.89; H, 4.89; N, 4.53; found C, 69.76; H, 4.91; N, 4.49. m/z: 309.10 (100.0%).

MR1076: (Z)-6-(4-((3-oxobenzofuran-2(3H)-ylidene)methyl)phenoxy)hexanoic acid

MR1076 was obtained by the condensation of benzofurane (1 eq) and ethyl 6-(4-formylphenoxy)hexanoate (1 eq) in Cholinechloride/urea (½) and catalytic amount of 50% KOH at 60° C. for 1 h.

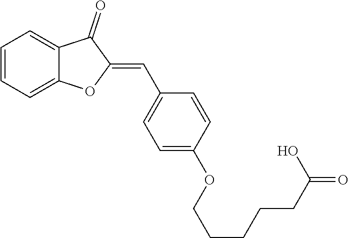 [SMILES: O=C(O)CCCCCOc1ccc(/C=C2\Oc3ccccc3C2=O)cc1]

Yield: 84%; 1H NMR (300 MHz, DMSO d6): δ 7.98-7.95 (d, 2H, J=8.9 Hz, C-H2′), 7.79 (m, 2H, C—H4,6), 7.58-7.55 (d, 1H, J=8.4 Hz, C—H7), 7.32 (t, 1H, J=7.5 Hz, C—H5), 7.09-7.06 (d, 2H, J=8.8 Hz, C—H3′), 6.95 (s, 1H, C—H10), 4.05 (t, 2H, (d, 2H, J=6.4 Hz, CH2□), 2.24 (t, 2H, J=7.0 Hz, CH2□), 1.74 (m, 2H, CH2□), 1.55 (m, 2H, CH2□), 1.43 (m, 2H, CH2□). 13C NMR (75 MHz, DMSO d6): δ 183.26 (C-3), 174.5 (COOH), 165.13 (C-8), 160.34 (C-4′), 145.12 (C-2), 137.3 (C-6), 133.44 (C-2′), 124.28 (C-4), 124.15 (C-5), 123.8 (C-1′), 121.16 (C-9), 115.15 (C-3′), 113.18 (C-7), 112.83 (C-10), 67.64 (C-a), 33.69 (C-e), 28.32 (C-b), 25.12(C-g), 24.29 (C-d).

Elemental analysis calcd (%) for C₂₁H₂O₅: C, 71.58; H, 5.72; found C, 71.44; H, 5.81. m/z: 352.13 (100.0%).

MR1120: (Z)-ethyl 2-(3,4-bis(benzyloxy)benzylidene)-3-oxo-2,3-dihydrobenzofuran-5-carboxylate

Starting from MR1065.

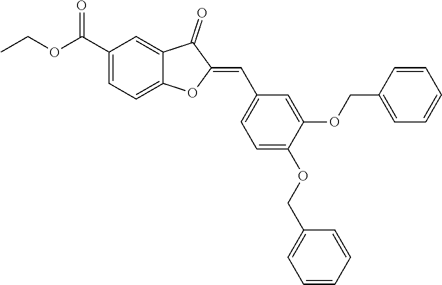 [SMILES: CCOC(=O)c1ccc2c(c1)C(=O)/C(=C/c1ccc(OCc3ccccc3)c(OCc3ccccc3)c1)O2]

Starting from MR1065.

Yield: 60%; 1H NMR (300 MHz, DMSO d6): δ 8.35-8.34-8.30 (dd, 1H, J=1.8;8.6 Hz, C—H6), 8.22 (d, 1H, J=1.4 Hz, C—H4), 7.72 (d, 1H, J=1.8 Hz, C-H2′), 7.66-7.63 (d, 1H, J=8.6 Hz, C—H7), 7.58-7.53 (dd, 1H, J=1.8; 8.6 Hz, C—H6′), 7.50-7.30 (m, 10H, C—H benzyl), 7.20-7.17 (d, 1H, J=8.6 Hz, C—H5′), 6.93 (s, 1H, C—H10), 5.23 (bs, 4H, CH2), 4.30 (q, 2H, OCH2), 1.29 (t, 3H CH3).

13C NMR (75 MHz, DMSO d6): δ 182.45(C-3), 165.24 (COOH), 166.2 (C-8), 150.53 (C-3′), 148.08 (C-4′), 145.49 (C-2), 137.98(C-6), 137.06 (C-1 bn), 136.74 (C-1bn), 128.49 (C-3bn), 127.97 (C-4bn), 127.9 (C-4bn), 127.66 (C-2bn), 127.58 (C-2bn), 126.54 (C-1′), 126.46 (C-4), 125.38 (C-6′), 124.52 (C-9), 121.37 (C-5), 116.77 (C-5′), 114.08 (C-7), 114.07 (C-2′), 113.58 (C-10), 70.08 (CH2), 69.92 (CH2), 60.61 (CH2), 14.23 (CH3).

MR1121: (Z)-decyl 2-(3,4-bis(benzyloxy)benzylidene)-3-oxo-2,3-dihydrobenzofuran-5-carboxylate

Starting from MR1065.

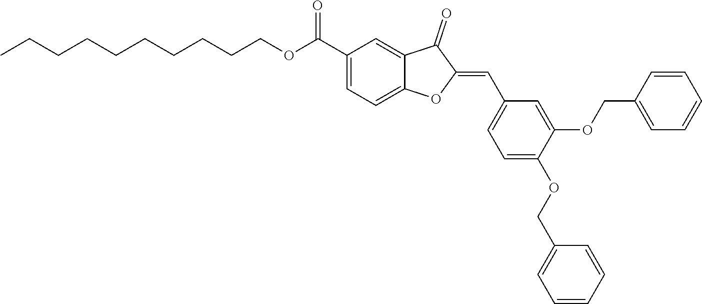 [SMILES: CCCCCCCCCCOC(=O)c1ccc2c(c1)C(=O)/C(=C/c1ccc(OCc3ccccc3)c(OCc3ccccc3)c1)O2]

Starting from MR1065.

Yield: 60%; 1H NMR (300 MHz, DMSO d6): δ 8.35-8.34-8.30 (dd, 1H, J=1.8; 8.6 Hz, C—H6), 8.22 (d, 1H, J=1.4 Hz, C—H4), 7.72 (d, 1H, J=1.8 Hz, C-H2′), 7.66-7.63 (d, 1H, J=8.6 Hz, C—H7), 7.58-7.53 (dd, 1H, J=1.8; 8.6 Hz, C—H6′), 7.50-7.30 (m, 10H, C—H benzyl), 7.20-7.17 (d, 1H, J=8.6 Hz, C—H5′), 6.93 (s, 1H, C—H10), 5.23 (bs, 4H, CH2), 4.56 (t, 2H, OCH2), 1.80 (m, 2H CH2), 1.43-1.29 (m, 14H), 0.83 (t, 3H, CH3).
[1] Reaction of 2′-hydroxy-5′-acetamido chalcones with dimethyl sulfoxide-iodine, pyridine-mercuric (II) acetate and triethanolamine. Agrawal, Nitin N.; Soni, P. A., Indian Journal of Chemistry, Section B: Organic Chemistry Including Medicinal Chemistry, 44B(12), 2601-2603; 2005.
[2] A new process for the synthesis of aurones by using mercury(II) acetate in pyridine and cupric bromide in dimethyl sulfoxide. Agrawal, Nitin N.; Soni, P. A., Indian Journal of Chemistry, Section B: Organic Chemistry Including Medicinal Chemistry. 45B(5), 1301-1303, 2006.

### Example B: Biology

### 1. Material and Methods

Reference strains used were obtained from either the American Type Culture Collection (ATCC, Molsheim Cedex France), the German Leibniz Institute (DSMZ, Braunschweig, Germany) or the French Pasteur Institute (CIP, Paris, France).

Antimicrobial Activity Assay

Most of the bacterial strains were routinely grown on Luria Bertani (LB) agar plates and LB broth at 37° C. in aerobic condition except H. pylori that was grown in BHI in micro-aerobic condition using micro-aerobic BD GasPak generator (Sigma-Aldrich, Lyon, France). M. smegmatis was cultured in Middlebrook 7H10 agar plate and Middlebrook 7H10 broth at 37° C. in aerobic condition. Clostridi, E. faecalis, P. acnes and S. pyogenes were cultured in Brain Heart Infusion (BHI) agar plates and BHI broth at 37° C. in anaerobic chamber (Coy Laboratory Products, Grass Lake, MI, USA).

Antimicrobial activity of aurones was evaluated by determination of their minimal inhibitory concentration (MIC) using two-fold serial dilutions in bacterial liquid media following the National Committee of Clinical Laboratory Standards (NCCLS, 1997). For most of the bacteria, single colonies of the different bacterial strains cultured on specific agar plates were used to inoculate 3 mL of Mueller-Hinton (MH), except Clostridi, P. acnes, H. pylori, E. faecalis, and S. pyogenes or M. smegmatis that were cultured in 3 mL of BHI or Middlebrook 7H10 broth, respectively. Tubes were then incubated overnight (for approximately 16 h) at 37° C. under stirring (200 rpm), except for M. smegmatis that was left to grow for 48-72 h. Optical density (OD) of the bacterial suspensions were then read at 600 nm, adjusted to 1 with medium before bacteria were diluted 1/100 in 3 mL of fresh medium and incubated at 37° C., 200 rpm until bacteria reached log phase growth (OD_{600 nm} around 0.6). In the case of Clostridi, P. acnes, B. thetaioataomicron, E. faecalis, H. pylori, S. pyogenes and M. smegmatis, MIC were performed directly using over-night growing suspension. In all cases, bacteria were diluted in appropriate medium to reach bacterial density around 10⁵ cells/mL. 100 μL per well of bacterial suspension were then added into sterile polypropylene 96 well microplates (Greiner BioOne, Dominique Dutscher, Brumath, France). Bacteria were exposed to increasing concentrations of aurones obtained by serial dilution (1:2 dilution). Plates were incubated at 37° C. for 18-24 h for all bacteria except M. smegmatis that where incubated for 72 h. All bacterial strains were tested in aerobic conditions except Clostridi, P. acnes, B. thetaioataomicron, E. faecalis, S. pyogenes that were incubated in an anaerobic chamber (Coy Laboratory Products, Grass Lake, MI, USA). MIC on H. pylori was performed in a micro-aerobic atmosphere generated using anaerobic BD GasPak system (Sigma-Aldrich, Lyon, France).

Antifungal Activity Assay

Fungal strains were grown on Potato Dextrose (PD) agar plates.

The antifungal effect of aurones was tested following the reference methods for yeasts (NCCLS M27-A) and molds (M38-P). Liquid suspension of C. albicans was prepared by resuspending colonies collected from LB plates in sterile NaCl 0.85% solution. C. albicans were then diluted at 1-2×10³ yeasts/mL in RMPI media buffered with MOPS (final concentration of 0.165 mol/L (pH 7.0)). For filamentous fungi, conidi were collected from fungi grown on PDA plates using sterile solution of NaCl 0.9% supplemented with Tween at 0.1%. After counting under microscope, dilution at 2-3×10⁴ conidi/mL were prepared in PD media or RMPI buffered with MOPS (final concentration of 0.165 mol/L (pH 7.0)). Diluted yeast or fungi were then subjected to MIC testing as described for bacteria. For C. albicans, plates were incubated at 35° C. for 24 h before reading. For other fungi, plates were incubated for 3 to 4 days before reading at room temperature. At the end of the incubation, OD_{600 nm} was measured using microplate reader (Biotek, Synergy Mx, Colmar, France). The MIC was defined as the lowest concentration of drug that inhibited visible growth of the organism. Experiments were conducted in independent triplicate (n=3).

Cytotoxic Assay on Human Cells

The toxicity of aurones on human cells was evaluated using resazurin assay. Cells used were: human kidney cell line A498 (ATCC® HTB-44), human normal lung epithelial cells BEAS-2B (ATCC® CRL-9609), human intestinal cell line Caco-2 (ATCC® HTB-37), human normal epidermal keratinocytes HaCaT (from Creative Bioarray, Shirley, NY 11967, USA), human liver cell line HepG2 (ATCC® HB-8065), human normal vascular endothelial cells HUVEC (obtained from ECACC, Sigma-Aldrich, Lyon, France), human normal lung fibroblast IMR90 cells (ATCC® CCL186) and human gastric cell line N87 (ATCC® CRL-5822). A498, BEAS-2B, Caco-2, HaCaT, HepG2 and IMR-90 cells were cultured in DMEM supplemented with 10% fetal calf serum (FCS), 1% 1-glutamine, and 1% antibiotics (all from Invitrogen (Carlsbad, CA, USA). HUVEC cells were cultured in specific medium (from Sigma Aldrich). N87 cells were cultured in RPMI media supplemented with 10% fetal calf serum (FCS), 1% L-glutamine, and 1% antibiotics (all from Invitrogen, Carlsbad, CA, USA). Cells were routinely grown on 25 cm² flasks and maintained in a 5% CO₂ incubator at 37° C. For toxicity assay, cells were detached using trypsin-EDTA solution (Thermo Fisher Scientific, Illkirch-Graffenstaden, France), counted using Mallasez counting chamber and seeded into 96-well cell culture plates (Greiner bio-one, Dominique Dutscher, Brumath, France) at approximately 10,000 cells per well. The cells were left to grow for 48-72 h at 37° C. in a 5% CO₂ incubator until they reached confluence. Media from wells was then aspirated and cells were treated with 100 μL of culture media containing increasing concentrations of aurones obtained by serial dilution (1:2 dilution), DMSO (0.5% DMSO final concentration) was used as negative control. After 48 h incubation at 37° C. in a 5% CO₂ incubator, cell viability was evaluated using resazurin based in vitro toxicity assay kit (Sigma-Aldrich, Lyon, France) following manufacturer's instructions. More particularly, cell wells were empty and cells were treated with 100 μL of resazurin diluted 1:10 in sterile PBS containing calcium and magnesium (PBS⁺⁺, pH 7.4). After 4 h incubation at 37° C., fluorescence intensity (excitation wavelength of 530 nm/emission wavelength of 590 nm) was measured using microplate reader (Biotek, Synergy Mx, Colmar, France). The fluorescence values were normalized by the controls (DMSO treated cells) and expressed as percent viability. The IC₅₀ values of aurones on cell viability (i.e. the concentration of derivative causing a reduction of 50% of the cell viability) were calculated using GraphPad® Prism 7 software (San Diego, CA, USA).

Selection for Resistant Bacteria.

To evaluate if compounds could induce or not bacterial resistance, cultures of B. mallei and B. pseudomallei were continuously exposed to tested compounds over 15 consecutive days. Broth microdilution susceptibility testing (MIC measurement) was performed using a standard doubling-dilution series of tested compounds concentrations on day 1. Following the incubation of the cultures for 24 h, and the determination of the MIC, the wells that contained the highest concentration of tested compounds permitting growth were diluted 1:1000 in MH and used to provide the inoculum for the next MIC assay, this process being repeated daily for 15 consecutive days. Doxycycline was used as control antibiotic known to cause induction of resistance in Burkholderia species.

Mutagenicity

Chemicals and Culture Medium

All reagents were handled and used in sterile conditions. They were purchased from Sigma-Aldrich.
1. Medium: Mc Coys'5A (storage at 4° C.)
2. Complete medium: McCoy's 5A medium supplemented with 10% fetal calf serum, 2 mM L-glutamine and a mixture of penicillin-streptomycin (100 U/ml—10 m/ml).
3. Exogenous metabolic fraction S9 mix: Extract from rat liver (S9, storage at —80° C.) 10%, 1M KCl, MgCl12 6H2)0.2 M, 0.2 M G6P, NADP 0.04 M in 0.2 M phosphate buffer (PBS)
4. Washing solution: phosphate buffer (PBS)
5. Blocking agent for cytokinesis: Cytochalasin B (stock solution: 1 mg/ml in DMSO, −80° C. storage)
6. Positive controls: Mitomycin C (Stock solution: 0.1 mg/ml in DMSO, storage −80° C.), benzo-a-pyrene (Stock solution: 1 mg/ml in DMSO, storage −80° C.)

Cell Line

The micronucleus assay was performed on a Chinese Hamster Ovary cell line CHO-K1 (ATCC, USA). This cell line was chosen for its rapid cell cycle (doubling time of 24 hours) and its genetic stability. It has been validated and accepted for the MNvit test by the OECD. Cells were maintained in McCoy's 5A medium supplemented with 10% fetal calf serum, 1 mM glutamine and 100-U/mL-10 μg·mL-1 of a mixture of penicillin-streptomycin. They were incubated at 37° C. in 5% CO₂.

Reference Elements

Solvent control: dimethyl sulfoxide DMSO (1%, v:v)

Positive controls: mitomycin C (0.6 μg/ml) and benzo-a-pyrene (5 μg/ml), diluted in DMSO and stored at −80° C.

Solubilization of Test Substances

The test substances were dissolved into dimethyl sulfoxide (DMSO). Stock solutions (10 mM) were stored at −80° C. in the dark. Then 1/10, 1/100 and 1/1000 dilutions of this stock solution were made for the assay.

Assay Protocol

All the assays were conducted in duplicate. The CHO-K1 cells, suspended in Mac Coys'5A medium, were transferred into Labteck wells at a concentration of 100,000 cells/ml, and incubated for 24 hours at 37° C. in CO₂ (5%).

When the test was performed without metabolic activation, the test substances were added into cell cultures at concentrations previously defined. A negative control containing culture medium, a solvent control containing 1% DMSO and a positive control containing 0.6 μg/ml of mitomycin C were added.

When the assay was performed in the presence of metabolic activation, S9 mix metabolizing mixture was added to cell cultures at a concentration of 10%. Then the test substances were added to the cell cultures at concentrations previously defined. A negative control containing culture medium, a solvent control containing 1% DMSO and a positive control containing 5 μg/ml of benzo-a-pyrene were added.

After 3 hours of incubation at 37° C. in CO₂ (5%), the culture medium was removed, the cells were rinsed with phosphate buffered saline (PBS), and then returned to culture in McCoy's 5A medium containing 3 μg/ml of cytochalasin B. After a 21-hour incubation period at 37° C., cells were rinsed with phosphate buffered saline (PBS), fixed with methanol and stained with 10% Giemsa for 20 minutes.

Analysis of Results

The analysis of results was performed under a microscope at ×1000 magnification. The antiproliferative activity of test substances was estimated by counting the number of binucleated cells relative to the number of mononucleated cells on a total of 500 cells for each dose (250 cells counted per well). The proliferation index (Cytokinesis Blocked Proliferative Index CBPI) was calculated using the following formula:

$$CBPI = \frac{2 ⋆ BI + MONO}{500}$$

BI: number of binucleated cells
MONO: number of mononucleated cells

The cytostasis index (CI %), i.e. the percentage of cell replication inhibition, was calculated using the following formula:

CI %: 100−{100×(CBPI_{test material}−1)/(CBPI_{solvent control}−1)

After this step, only the doses inducing a decrease of less than 55±5% of CI % as compared to the negative control were taken into account for counting micronuclei.

The rates of micronuclei were evaluated for the presence of independent nuclear core entities in 1000 binucleated cells per well, which corresponds to 2000 cells examined by test substance dose. Micronuclei were identified as small nuclei well differentiated from cell nucleus, stained in the same manner and having a diameter less than one third of that of the cell nucleus.

Micronuclei rates obtained for different doses of test substances were compared to the negative control by a λ2 test. The assay was considered positive if:
A dose-response relationship was obtained between the rate of micronuclei and the doses tested,
At least one of these doses induced a statistically significant increase (P<0.05) in the number of micronucleated cells as compared to the negative control.

### 2. Results

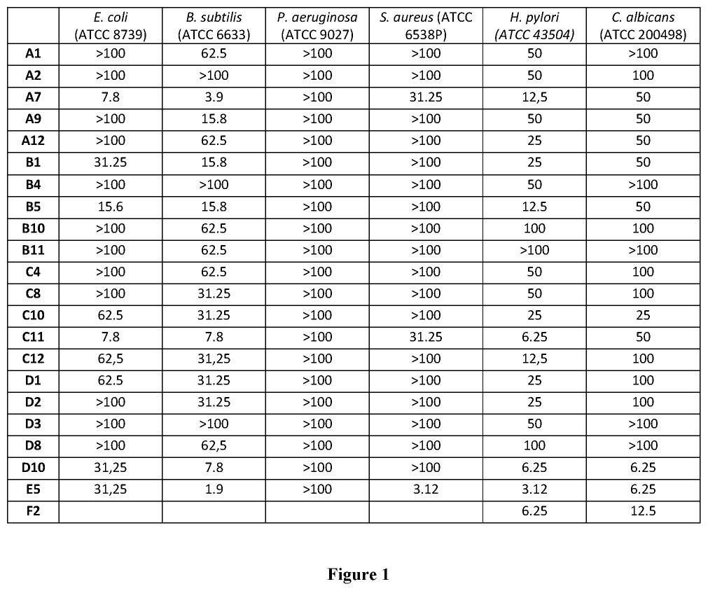

The antimicrobial and antifungal effects of the compounds according to the invention on reference bacterial and fungal strains are presented in the Tables of FIGS. 1,2, and 10. Minimal Inhibitory Concentration (MIC) values are expressed in μM.

In FIG. 1, the antimicrobial effect of compounds was tested against Gram⁻ bacteria (E. coli, P. aeruginosa and H. pylori), Gram⁺ bacteria (B. subtilis and S. aureus) and fungal strain (C. albicans). Measured MIC values ranged from 1.9 μM to >100 μM. Compounds A7, B5, C11, C12 and E5 were found the more active on tested strains.

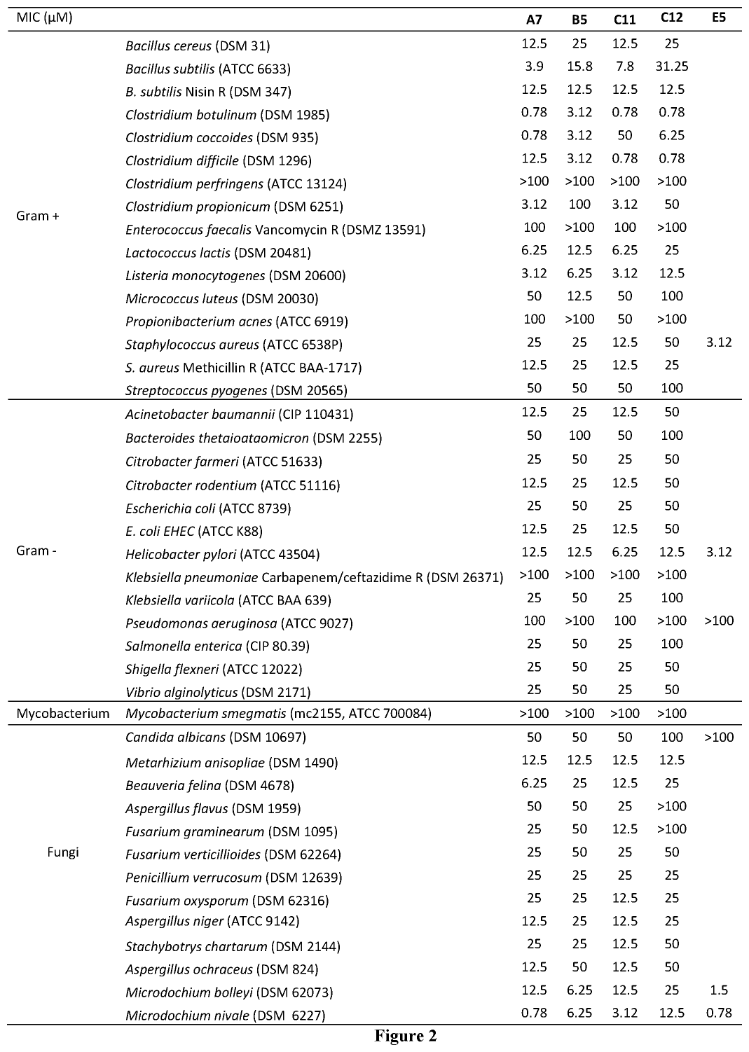

In FIG. 2, the antimicrobial effect of compounds A7, B5, C11, C12 and E5 was further tested using a large panel of bacteria including Gram⁺ and Gram⁻ bacteria and Mycobacterium as well as fungi associated to plant infection. Results confirmed the activity of compounds A7, B5, C11, C12 and E5 against almost all strains tested, suggesting a large spectrum of activity. Particularly low MIC values (as low as 0.78-1.5 μM) were found with Clostridium botulinum, C. difficile, Microdochium bolleyi, and M. nivale.

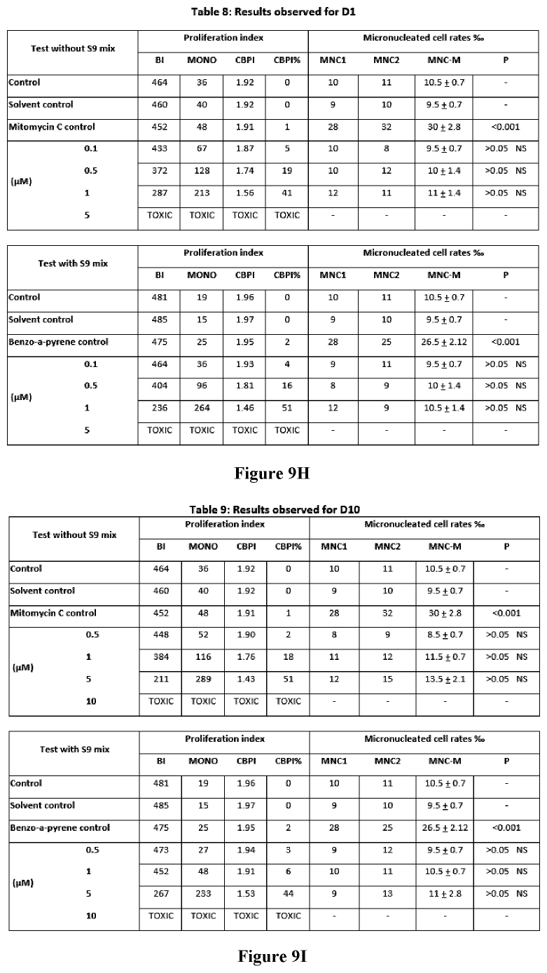

In FIG. 10, the antimicrobial effect of other compounds, namely AD-1-49, AD-1-61, AD-1-62, MR1065 and MR1076 was tested against Gram⁻ bacteria (E. coli, P. aeruginosa), Gram⁺ bacteria (B. subtilis, S. aureus and C. difficile) and fungal strain (C. albicans). Measured MIC values ranged from 3.12 μM to >100 μM. Interestingly compound MR1076 was found to be active on P. aeruginosa.

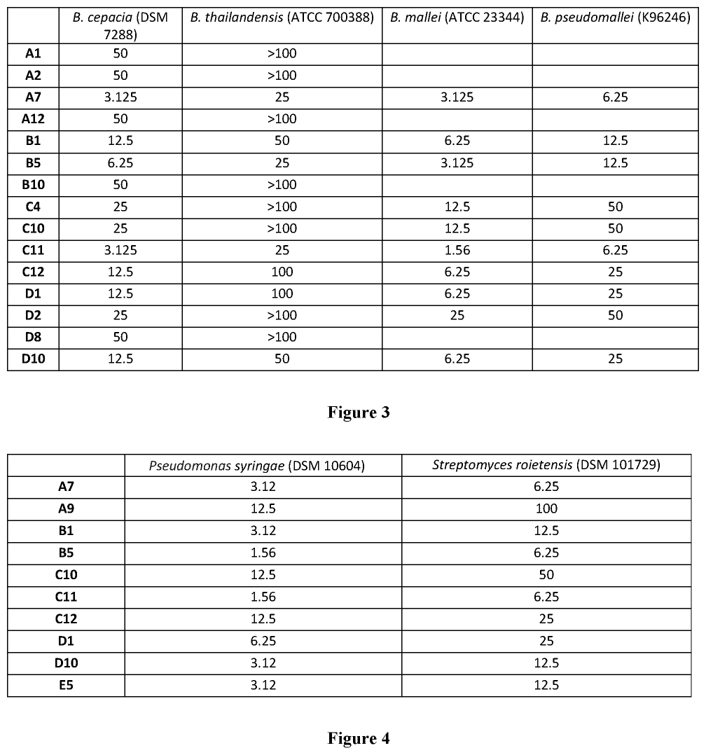

The antimicrobial of the compounds according to the invention on Burkholderia stains is presented in the Table of FIG. 3. Minimal Inhibitory Concentration (MIC) values are expressed in μM.

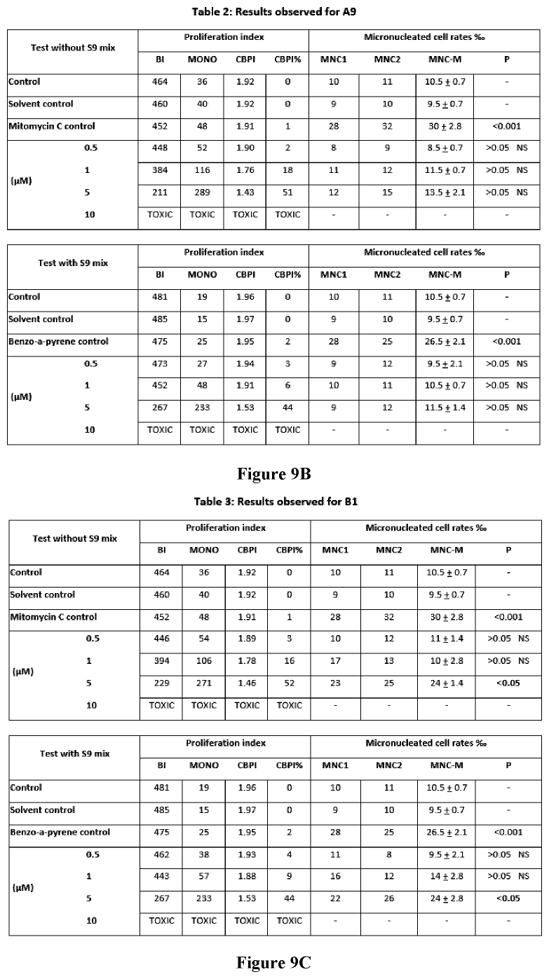

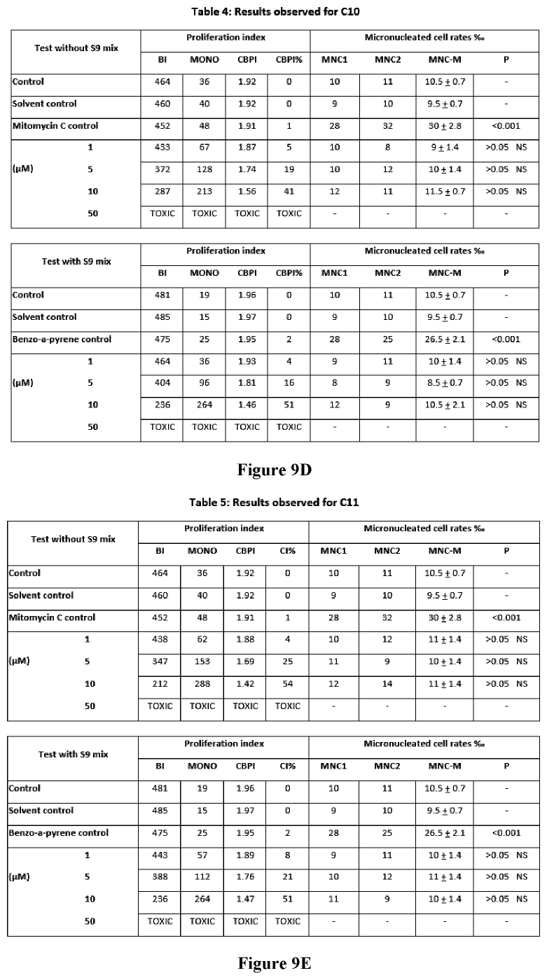

FIG. 3 shows that compounds are actives against different strains of Burkholderia causing infections in humans and animals. In addition, Burkholderia species are known to be resistant to numerous antibiotics used in medicine including aminoglycosides, beta-lactams and polymyxins. B. thailandensis that only rarely causes disease in humans or animals was found the less sensitive to compounds tested with MIC values ranging from 25 to >100 μM. B. cepacia causing lung infection in patient suffering from cystic fibrosis was found sensitive to tested compounds, particularly compounds A7, B 1, B5, C11, C12, D1 and D10 with MIC ranging from 3.12 to 12.5 μM for these compounds. B. mallei and B. pseudomallei that are classified as biological warfare agents and cause glanders and melioidosis, also known as Whitmore's disease, an infectious disease present in humans and animals (particularly horses) are also sensitive to tested compounds, particularly compounds A7, B1, B5, C4, C10, C11, C12, D1 and D10 with MIC values ranging from 1.5 to 12.5 μM for these compounds. Importantly, evaluation of the induction of resistance on B. mallei and B. pseudomallei by compounds A7, B1, B5, C4, C10, C11, C12, D1, D2 and D10 revealed that tested compounds caused minor to no resistance over 15 days of continuous exposure. Oppositely, the control antibiotic doxycycline caused the appearance of a progressive resistance reaching a 32-folds increase in its MIC after 13-15 days of continuous exposure (FIGS. 7 and 8).

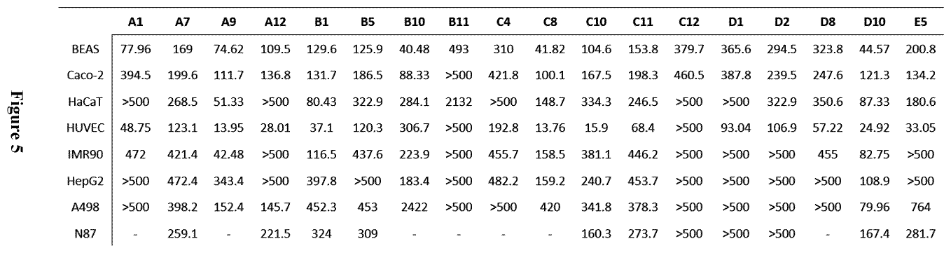

The antimicrobial of the compounds according to the invention on bacterial plant pathogens is presented in the Table of FIG. 4. Minimal Inhibitory Concentration (MIC) values are expressed in μM.

FIG. 4 shows that tested compounds are active both of Gram⁻ (Pseudomonas syringae) and Gram⁺ (Streptomyces roietensis) plant pathogens. Best compounds were compounds A7, B1, B5, C11, D10 and E5 with MIC values ranging from 1.5 to 3.12 μM on P. syringae and compounds A7, B1, B5, C11, D10 and E5 with MIC values ranging from 6.25 to 12.5 μM on S. roietensis.

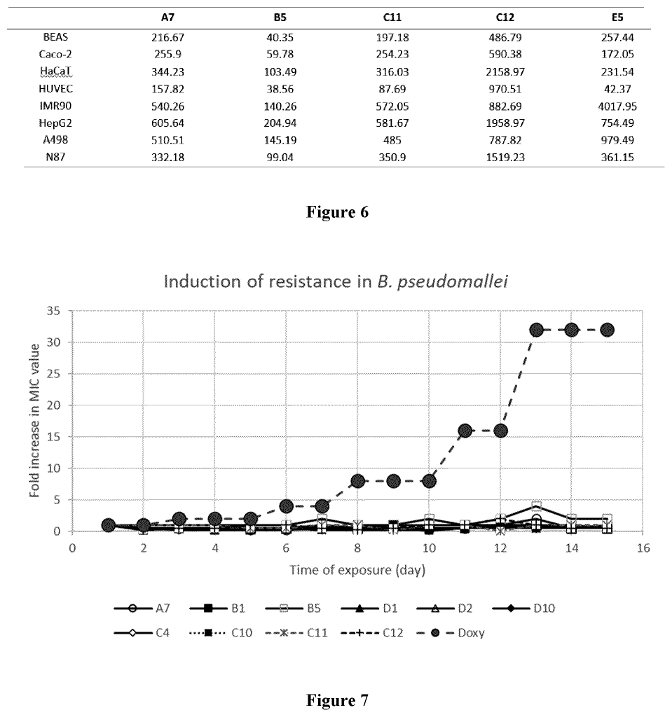

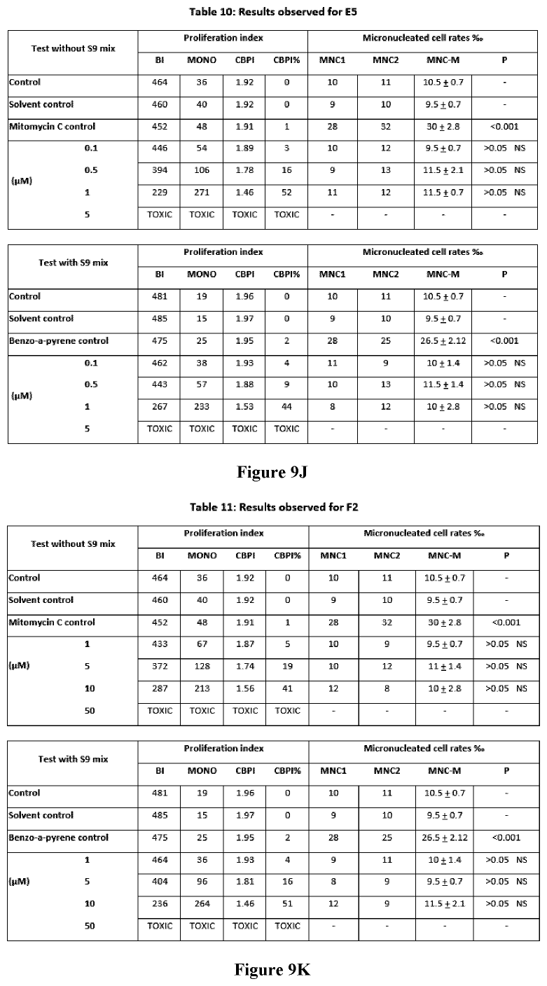

The Evaluation of the toxicity of the compounds according to the invention on different human cells is presented in the Table of FIG. 5 and FIG. 11.

FIG. 5 gives the IC50 of the tested compounds on the viability of human cells originating from different tissues/organs (i.e. the concentration of compound causing a reduction of 50% of the cell viability). Data demonstrate that compounds A7, B5, C11, C12 and E5 are the safer with IC50 higher than 100 μM in most cases and even higher than 500 μM (the highest dose tested).

FIG. 11 gives the IC50 (i.e. the concentration of compound causing a reduction of 50% of the cell viability) of the tested compounds AD-1-49, AD-1-61, AD-1-62, MR1065 and MR1076 on the viability of HepG2 cells originating from human liver. Data demonstrate that all five compounds are safe with IC50 higher than 250 μM in all cases and even higher than 1000 μM as far as MR1065 is concerned.

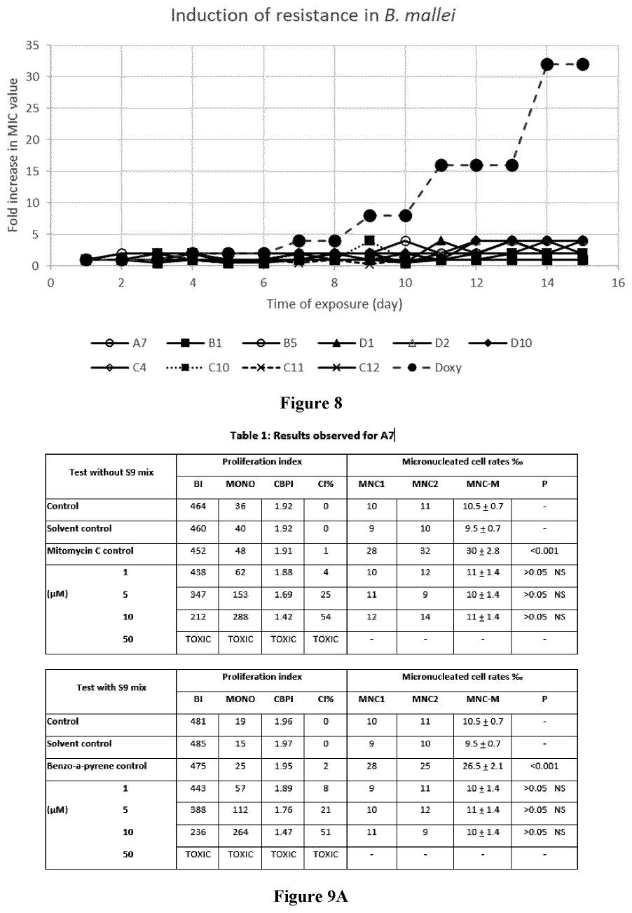

The therapeutic index or safety factor of preferred compounds according to the invention A7, B5, C11, C12, and E5 is presented in the Table of FIG. 6. Therapeutic index (TI) or safe factor (SF) were calculated by dividing the IC50 obtained for each cell type by the lowest MIC values observed for the same compound.

FIG. 6 clearly shows that although safety values depend both of the compound tested and cell type considered, compounds A7, B5, C11, C12 and E5 are very safe with TI/SF ranging from 38 to 4017. Based on the mean of TI/SF values of each tested compounds obtained on the different cell types considered, it appears that the order of safety of compounds is as follow C12 (mean of TI/SF=1169)>E5 (mean of TI/SF=852)>A7 ‘(mean of TI/SF=370)>C11 (mean of TI/SF=355)>B5 (mean of TI/SF=103).

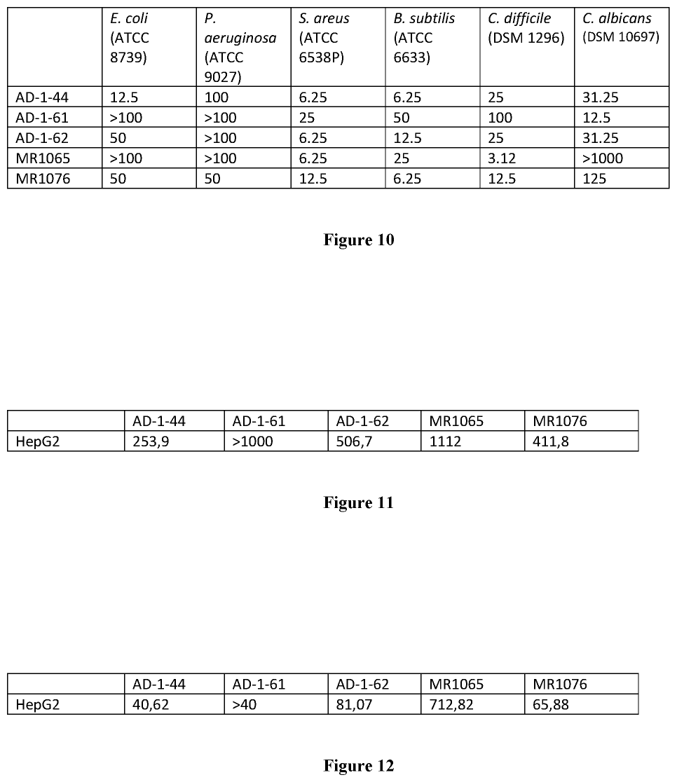

The therapeutic index or safety factor of compounds according to the invention AD-1-49, AD-1-61, AD-1-62, MR1065 and MR1076 is also presented in the Table of FIG. 12. Therapeutic index (TI) or safe factor (SF) were calculated by dividing the IC50 obtained for each cell type by the lowest MIC values observed for the same compound. Interestingly, compound MR1065 appears to have the best therapeutic index (712.82).

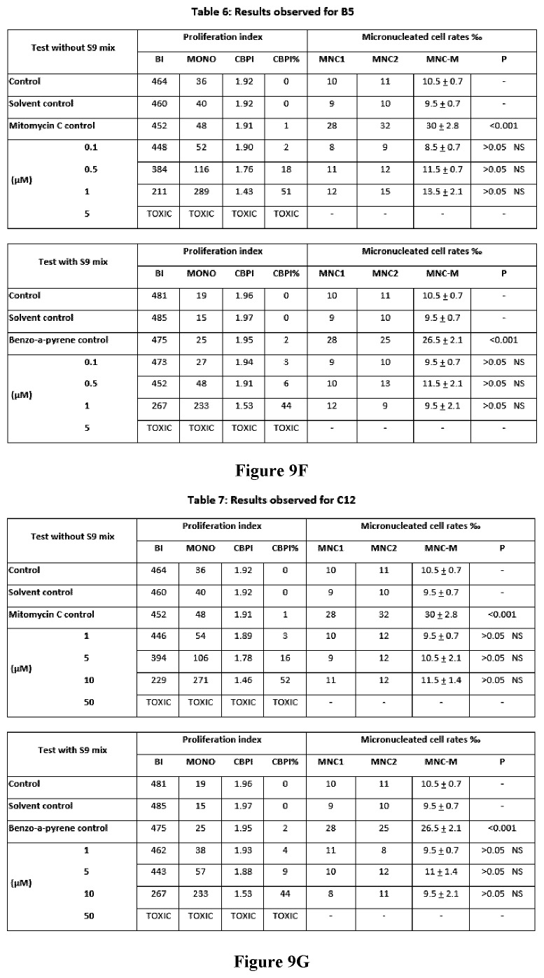

Results on mutagenicity are presented in tables 1-11 in FIGS. 9A-K. Results show that none of compounds induced a significant increase of micronucleated cells.

1-17. (canceled)

18. A compound of formula (I), the salts and the stereoisomers thereof: 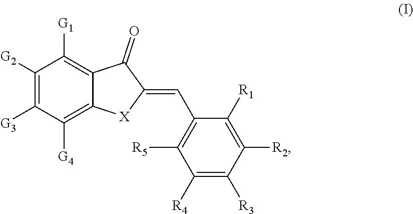
wherein:
X represents O, NH, S, or CH₂—;
G₁ represents a hydrogen or a (C₁-C₆)alkyloxy;
G₂ represents a radical selected from the group consisting of:
a hydrogen;
a (C₁-C₆)alkyl optionally substituted by a —NR₆R₇ group with R₆ and R₇ being independently a hydrogen or a —COR₈ with R₈ being a (C₁-C₆)alkyl;
a —NR₆R₇ group with R₆ and R₇ being independently a hydrogen, a —COR₈ with R₈ being a (C₁-C₆)alkyl, or an arginine residue;
a carbamimidoyl-guanidine; and
a —COOR₉ with R₉ being a hydrogen, a (C₁-C₁₂)alkyl, a geranyl, a farnesyl or a citronnellyl;
G₃ represents a radical selected from the group consisting of:
a hydrogen;
a hydroxy;
a —NR₆R₇ group with R₆ and R₇ being independently a hydrogen, a —COR₈ with R₈ being a (C₁-C₆)alkyl, or an arginine residue; and
a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
with the proviso that when G₂ and G₃ represent a hydrogen, then:
R₁ and R₃ represent a benzyloxy; or
R₃ represents a —O-flavone optionally substituted by at least a (C₁-C₁₀)alkyloxy or a hydroxy, or a —CH₂-berberine; and
with the proviso that said compound of formula (I) is not a compound selected from the group consisting of:
(Z)-N-(2-(4-methoxybenzydene)-3-oxo-2,3-dihydrobenzofuran-5-yl)acetamide;
(Z)-N-(3-oxo-2-(3,4,5-trimethoxybenzylidene)-2,3-dihydrobenzofuran-5-yl)acetamide; and
(Z)-N-(2-benzylidene-3-oxo-2,3-dihydrobenzofuran-5-yl)acetamide.

19. The compound according to claim 18, wherein R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl.

20. The compound according to claim 18, wherein:
G₂ represents a hydrogen, or a —NR₆R₇ group with R₆ and R₇ being independently a hydrogen or a —COR₈ with R₈ being a (C₁-C₆)alkyl; and
G₃ represents a hydrogen, or a —NR₆R₇ group with R₆ and R₇ being independently a hydrogen or a —COR₈ with R₈ being a (C₁-C₆)alkyl;
with the proviso that when one of G₂ and G₃ represents a —NR₆R₇ group with R₆ and R₇ being independently a hydrogen or a —COR₈ with R₈ being a (C₁-C₆)alkyl, then the other represents a hydrogen.

21. The compound according to claim 18, wherein R₃ represents a —O-flavone optionally substituted by at least a (C₁-C₁₀)alkyloxy or a hydroxy, or a —CH₂-berberine.

22. The compound according to claim 18, wherein G₂ represents COOH.

23. The compound according to claim 18, wherein R₃ and R₄ represent a benzyloxy.

24. The compound according to claim 18, wherein said compound is selected from the group consisting of:
A1: (Z)-N-(2-(2′-methoxybenzylidene)-3-oxo-2,3-dihydrobenzofuran-5-yl)acetamide;
A2: (Z)-N-(2-(3-methoxybenzylidene)-3-oxo-2,3-dihydrobenzofuran-5-yl)acetamide;
A7: (Z)-N-(2-(3-(benzyloxy)benzylidene)-3-oxo-2,3-dihydrobenzofuran-5-yl)acetamide;
A9: (Z)-5-amino-2-(3-(benzyloxy)benzylidene)benzofuran-3(2H)-one;
A12: (Z)-N-(3-oxo-2-(4-phenoxybenzylidene)-2,3-dihydrobenzofuran-5-yl)acetamide;
B1: (Z)-5-amino-2-(3-phenoxybenzylidene)benzofuran-3(2H)-one;
B4: (Z)-N-(2-(3-isopropoxybenzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
B10: (Z)-N-(2-(3-fluorobenzylidene)-3-oxo-2,3-dihydrobenzofuran-5-yl)acetamide;
B11: (Z)-5-amino-2-(2-fluorobenzylidene)benzofuran-3(2H)-one;
C4: (Z)-4-((5-acetamido-3-oxobenzofuran-2(3H)-ylidene)methyl)benzoic acid;
C8: (Z)-N-(7-nitro-3-oxo-2-(3-phenoxybenzylidene)-2,3-dihydrobenzofuran-5-yl)acetamide;
C10: (Z)-2-benzylidene-6-hydroxy-4-methoxy-5,7-dimethylbenzofuran-3(2H)-one;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
D2: (Z)-5-amino-2-(3,4,5-trimethoxybenzylidene)benzofuran-3(2H)-one;
D3: (Z)-N-(3-oxo-2-(2,4,5-trimethoxybenzylidene)-2,3-dihydrobenzofuran-5-yl)acetamide;
E5: (Z)-6-(4-((3-oxobenzofuran-2(3H)-ylidene)methyl)phenoxy)-2-phenyl-4H-chromen-4-one;
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.0^{2,10}.0^{4,8}.0^{15,20}]henicosa-1(21),2,4(8),9,13,15(20),16,18-octaen-13-ylium bromide;
AD-1-44: (Z)-N-(2-(3,4-bis(benzyloxy)benzylidene)-3-oxo-2,3-dihydrobenzofuran-5-yl)acetamide;
MR1065: (Z)-2-(3,4-bis(benzyloxy)benzylidene)-3-oxo-2,3-dihydrobenzofuran-5-carboxylic acid;
MR1076: (Z)-6-(4-((3-oxobenzofuran-2(3H)-ylidene)methyl)phenoxy)hexanoic acid;
MR1120: (Z)-ethyl 2-(3,4-bis(benzyloxy)benzylidene)-3-oxo-2,3-dihydrobenzofuran-5-carboxylate; and
MR1121: (Z)-decyl 2-(3,4-bis(benzyloxy)benzylidene)-3-oxo-2,3-dihydrobenzofuran-5-carboxylate.

25. A phytoprotective or decontaminating or disinfectant composition comprising a compound of formula (I), the salts and the stereoisomers thereof: 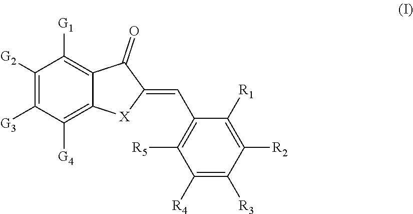
wherein:
X represents O, NH, S, or CH₂—;
G₁ represents a hydrogen or a (C₁-C₆)alkyloxy;
G₂ represents a radical selected from the group consisting of:
a hydrogen;
a (C₁-C₆)alkyl optionally substituted by a —NR₆R₇ group with R₆ and R₇ being independently a hydrogen or a —COR₈ with R₈ being a (C₁-C₆)alkyl;
a —NR₆R₇ group with R₆ and R₇ being independently a hydrogen, a —COR₈ with R₈ being a (C₁-C₆)alkyl, or an arginine residue;
a carbamimidoyl-guanidine; and
a —COOR₉ with R₉ being a hydrogen, a (C₁-C₁₂)alkyl, a geranyl, a farnesyl or a citronnellyl;
G₃ represents a radical selected from the group consisting of:
a hydrogen;
a hydroxy;
a —NR₆R₇ group with R₆ and R₇ being independently a hydrogen, a —COR₈ with R₈ being a (C₁-C₆)alkyl, or an arginine residue; and
a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
with the proviso that when G₂ and G₃ represent a hydrogen, then:
R₁ and R₃ represent a benzyloxy; or
R₃ represents a —O-flavone optionally substituted by at least a (C₁-C₁₀)alkyloxy or a hydroxy, or a —CH₂-berberine;
or a compound D10: (Z)-2-(4-methoxybenzylidene)benzofuran-3(2H)-one, said composition optionally further comprising an anti-microbial and/or anti-fungal agent.

26. A process for the decontamination or the disinfection of a surface or an inert object comprising a step of applying a phytoprotective or decontaminating or disinfectant composition according to claim 25 to a surface or an inert object infested or suspected to be infested by a bacterium and/or a fungus.

27. A kit comprising a compound of formula (I), the salts and the stereoisomers thereof: 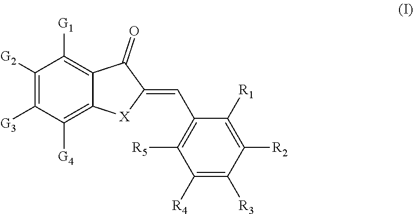
wherein:
X represents O, NH, S, or CH₂—;
G₁ represents a hydrogen or a (C₁-C₆)alkyloxy;
G₂ represents a radical selected from the group consisting of:
a hydrogen;
a (C₁-C₆)alkyl optionally substituted by a —NR₆R₇ group with R₆ and R₇ being independently a hydrogen or a —COR₈ with R₈ being a (C₁-C₆)alkyl;
a —NR₆R₇ group with R₆ and R₇ being independently a hydrogen, a —COR₈ with R₈ being a (C₁-C₆)alkyl, or an arginine residue;
a carbamimidoyl-guanidine; and
a —COOR₉ with R₉ being a hydrogen, a (C₁-C₁₂)alkyl, a geranyl, a farnesyl or a citronnellyl;
G₃ represents a radical selected from the group consisting of:
a hydrogen;
a hydroxy;
a —NR₆R₇ group with R₆ and R₇ being independently a hydrogen, a —COR₈ with R₈ being a (C₁-C₆)alkyl, or an arginine residue; and
a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
with the proviso that when G₂ and G₃ represent a hydrogen, then:
R₁ and R₃ represent a benzyloxy; or
R₃ represents a —O-flavone optionally substituted by at least a (C₁-C₁₀)alkyloxy or a hydroxy, or a —CH₂-berberine;
or a compound D10: (Z)-2-(4-methoxybenzylidene)benzofuran-3(2H)-one, and a further anti-microbial and/or anti-fungal agent as a combined preparation for simultaneous, separate, or sequential use for controlling a bacterium and/or a fungus.

28. A method of treating a bacterial infection and/or a fungal infection comprising administering a compound of formula (I), the salts and the stereoisomers thereof: 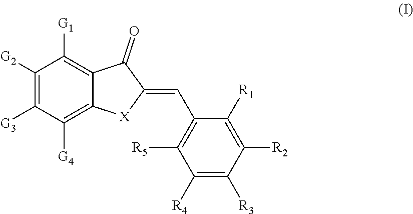
wherein:
X represents O, NH, S, or CH₂—;
G₁ represents a hydrogen or a (C₁-C₆)alkyloxy;
G₂ represents a radical selected from the group consisting of:
a hydrogen;
a (C₁-C₆)alkyl optionally substituted by a —NR₆R₇ group with R₆ and R₇ being independently a hydrogen or a —COR₈ with R₈ being a (C₁-C₆)alkyl;
a —NR₆R₇ group with R₆ and R₇ being independently a hydrogen, a —COR₈ with R₈ being a (C₁-C₆)alkyl, or an arginine residue;
a carbamimidoyl-guanidine; and
a —COOR₉ with R₉ being a hydrogen, a (C₁-C₁₂)alkyl, a geranyl, a farnesyl or a citronnellyl;
G₃ represents a radical selected from the group consisting of:
a hydrogen;
a hydroxy;
a —NR₆R₇ group with R₆ and R₇ being independently a hydrogen, a —COR₈ with R₈ being a (C₁-C₆)alkyl, or an arginine residue; and
a carbamimidoyl-guanidine;
G₄ represents a hydrogen, a nitro, or a (C₁-C₆)alkyl;
R₁ represents a hydrogen, a halogen, a (C₁-C₆)alkyloxy, a benzyloxy, or a phenyloxy;
R₂ represents a hydrogen, a halogen, a (C₁-C₆)alkyloxy, a phenyloxy, or a benzyloxy;
R₃ represents a hydrogen, a (C₁-C₆)alkyloxy, a —COOH, a phenyloxy, a benzyloxy, a —O-(C₁-C₆)alkyl-COOH, a —NR₁₀R₁₁ with R₁₀ and R₁₁ being independently a hydrogen or a (C₁-C₆)alkyl, a —O-flavone optionally substituted by at least a (C₁-C₁₀)alkyloxy or a hydroxy, or a —CH₂-berberine;
R₄ represents a hydrogen, a halogen, a (C₁-C₆)alkyloxy, a phenyloxy, or a benzyloxy; and
R₅ represents a hydrogen, a halogen, a (C₁-C₆)alkyloxy, a benzyloxy, or a phenyloxy;
with the proviso that when G₂ and G₃ represent a hydrogen, then:
R₁ and R₃ represent a benzyloxy; or
R₃ represents a —O-flavone optionally substituted by at least a (C₁-C₁₀)alkyloxy or a hydroxy, or a —CH₂-berberine;
or a compound D10: (Z)-2-(4-methoxybenzylidene)benzofuran-3(2H)-one;
to a subject in need of treatment.

29. The method according to claim 28, wherein said compound is selected from the group consisting of:
A1: (Z)-N-(2-(2′-methoxybenzylidene)-3-oxo-2,3-dihydrobenzofuran-5-yl)acetamide;
A2: (Z)-N-(2-(3-methoxybenzylidene)-3-oxo-2,3-dihydrobenzofuran-5-yl)acetamide;
A7: (Z)-N-(2-(3-(benzyloxy)benzylidene)-3-oxo-2,3-dihydrobenzofuran-5-yl)acetamide;
A9: (Z)-5-amino-2-(3-(benzyloxy)benzylidene)benzofuran-3(2H)-one;
A12: (Z)-N-(3-oxo-2-(4-phenoxybenzylidene)-2,3-dihydrobenzofuran-5-yl)acetamide;
B1: (Z)-5-amino-2-(3-phenoxybenzylidene)benzofuran-3(2H)-one;
B4: (Z)-N-(2-(3-isopropoxybenzylidene)-3-oxo-2,3-dihydrobenzofuran-5-yl)acetamide;
B5: (Z)-N-(2-(4-isopropoxybenzylidene)-3-oxo-2,3-dihydrobenzofuran-5-yl)acetamide;
B10: (Z)-N-(2-(3-fluorobenzylidene)-3-oxo-2,3-dihydrobenzofuran-5-yl)acetamide;
B11: (Z)-5-amino-2-(2-fluorobenzylidene)benzofuran-3(2H)-one;
C4: (Z)-4-((5-acetamido-3-oxobenzofuran-2(3H)-ylidene)methyl)benzoic acid;
C8: (Z)-N-(7-nitro-3-oxo-2-(3-phenoxybenzylidene)-2,3-dihydrobenzofuran-5-yl)acetamide;
C10: (Z)-2-benzylidene-6-hydroxy-4-methoxy-5,7-dimethylbenzofuran-3(2H)-one;
C11: (Z)-6-amino-2-(4-methoxybenzylidene)benzofuran-3(2H)-one;
C12: (Z)-2-(2,4-bis(benzyloxy)benzylidene)benzofuran-3(2H)-one;
D1: (Z)-N-(3-oxo-2-(3,4,5-trimethoxybenzylidene)-2,3-dihydrobenzofuran-5-yl)acetamide;
D2: (Z)-5-amino-2-(3,4,5-trimethoxybenzylidene)benzofuran-3(2H)-one;
D3: (Z)-N-(3-oxo-2-(2,4,5-trimethoxybenzylidene)-2,3-dihydrobenzofuran-5-yl)acetamide;
D8: (Z)-N-(2-benzylidene-3-oxo-2,3-dihydrobenzofuran-5-yl)acetamide;
D10: (Z)-2-(4-methoxybenzylidene)benzofuran-3(2H)-one;
E5: (Z)-6-(4-((3-oxobenzofuran-2(3H)-ylidene)methyl)phenoxy)-2-phenyl-4H-chromen-4-one;
F2: 16,17-dimethoxy-21-[(4-{[(2Z)-3-oxo-2,3-dihydro-1-benzofuran-2-ylidene]methyl}phenyl)methyl]-5,7-dioxa-13lambda5-azapentacyclo[11.8.0.02,10.04,8.015,20]henicosa-1(21),2,4(8),9,13,15(20),16,18-octaen-13-ylium bromide;
AD-1-44: (Z)-N-(2-(3,4-bis(benzyloxy)benzylidene)-3-oxo-2,3-dihydrobenzofuran-5-yl)acetamide;
AD-1-61: (2Z)-2-(3-methoxybenzylidene)-3-oxo-2,3-dihydro-1-benzofuran-5-carboxylic acid;
AD-1-62: (2Z)-2-[4-(dimethylamino)benzylidene]-3-oxo-2,3-dihydro-1-benzofuran-5-carboxylic acid;
MR1065: (Z)-2-(3,4-bis(benzyloxy)benzylidene)-3-oxo-2,3-dihydrobenzofuran-5-carboxylic acid;
MR1076: (Z)-6-(4-((3-oxobenzofuran-2(3H)-ylidene)methyl)phenoxy)hexanoic acid;
MR1120: (Z)-ethyl 2-(3,4-bis(benzyloxy)benzylidene)-3-oxo-2,3-dihydrobenzofuran-5-carboxylate; and
MR1121: (Z)-decyl 2-(3,4-bis(benzyloxy)benzylidene)-3-oxo-2,3-dihydrobenzofuran-5-carboxylate.

30. The method according to claim 28, wherein said bacterium is a Gram positive bacterium, Gram negative bacterium, or a Mycobacterium.

31. The method according to claim 30, wherein said Gram positive bacterium is selected from the group consisting of: Bacillus cereus, Bacillus subtilis, Clostridium botulinum, Clostridium coccoides, Clostridium difficile, Clostridium perfringens, Clostridium propionicum, Enterococcus faecalis, Lactococcus lactis, Listeria monocytogenes, Micrococcus luteus, Propionibacterium acnes, Staphylococcus aureus, Streptococcus pyogenes, and Streptomyces roietensis.

32. The method according to claim 30, wherein said Gram negative bacterium is selected from the group consisting of: Acinetobacter baumannii, Bacteroides thetaioataomicron, Burkholderia cepacia, Burkholderia thailandensis, Burkholderia mallei, Burkholderia pseudomallei, Citrobacter farmer, Citrobacter rodentium, Escherichia coli, E. coli EHEC, Helicobacter pylori, Klebsiella pneumoniae, Klebsiella variicola, Pseudomonas aeruginosa, Pseudomonas syringae, Salmonella enterica, Shigella flexneri, and Vibrio alginolyticus.

33. The method according to claim 30, wherein said Gram positive bacterium is an antibiotic resistant Gram positive bacterium selected from the group consisting of: B. subtilis Nisin R, and S. aureus Methicillin R, or an antibiotic resistant Gram negative bacterium selected from the group consisting of: Burkholderia cepacia, Burkholderia thai, Burkholderia mallei, and Burkholderia pseudomallei.

34. The method according to claim 28, wherein said fungus is selected from the group consisting of: Candida albicans, Metarhizium anisopliae, Beauveria feline, Aspergillus flavus, Fusarium graminearum, Fusarium verticillioides, Penicillium verrucosum, Fusarium oxysporum, Aspergillus niger, Stachybotrys chartarum, Aspergillus ochraceus, Microdochium bolleyi, Microdochium majus, and Microdochium nivale.
